(19) United States
(12) Patent Application Publication
(10) Pub. No.: US 2024/0285624 A1
(43) Pub. Date: Aug. 29, 2024

(54) ERK1/2 OR SHP2 INHIBITORS AND FLT3 INHIBITORS COMBINATION THERAPY

(71) Applicant: Erasca, Inc., San Diego, CA (US)

(72) Inventors: Robert Field SHOEMAKER, San Diego, CA (US); Wei LIN, San Diego, CA (US); Erin Denise LEW, San Diego, CA (US); Jingchuan ZHANG, San Diego, CA (US); Joanne OH, San Diego, CA (US); Leenus MARTIN, San Diego, CA (US); Leslie Harris BRAIL, San Diego, CA (US)

(21) Appl. No.: 18/570,831

(22) PCT Filed: Jun. 23, 2022
(86) PCT No.: PCT/US2022/034680

§ 371 Date: Dec. 15, 2023

Related U.S. Application Data
(60) Provisional application No. 63/321,616, filed on Mar. 18, 2022.
(60) Provisional application No. 63/321,608, filed on Mar. 18, 2022.
(60) Provisional application No. 63/314,659, filed on Feb. 28, 2022.
(60) Provisional application No. 63/314,653, filed on Feb. 28, 2022.
(60) Provisional application No. 63/214,766, filed on Jun. 24, 2021.
(60) Provisional application No. 63/214,763, filed on Jun. 24, 2021.

(51) Int. Cl. A61K 31/506; A61K 31/497

(52) U.S. Cl. CPC A61K 31/506; A61K 31/497

(57) ABSTRACT

The present disclosure relates generally to the use of an ERK1/2 inhibitor or a SHP2 inhibitor in combination with a FLT3 inhibitor, such as gilteritinib, for treating cancer, specifically acute myeloid leukemia (AML).

### CROSS-REFERENCE

This application claims the benefit of U.S. Provisional Application Ser. No. 63/214,766 filed Jun. 24, 2021, U.S. Provisional Application Ser. No. 63/214,763 filed Jun. 24, 2021, U.S. Provisional Application Ser. No. 63/314,653 filed Feb. 28, 2022, U.S. Provisional Application Ser. No. 63/314,659 filed Feb. 28, 2022, U.S. Provisional Application Ser. No. 63/321,608 filed Mar. 18, 2022, and U.S. Provisional Application Ser. No. 63/321,616 filed Mar. 18, 2022, which are hereby incorporated by reference in their entirety.

### BACKGROUND

ERK1 and ERK2 (collectively “ERK1/2”) are related protein-serine/threonine kinases that participate in, amongst others, the Ras-Raf-MEK-ERK signal transduction pathway, which is sometimes denoted as the mitogen-activated protein kinase (MAPK) pathway. This pathway is thought to play a central role in regulating a number of fundamental cellular processes including one or more of cell proliferation, survival, adhesion, cycle progression, migration, differentiation, metabolism, and transcription. The activation of the MAPK pathway has been reported in numerous tumor types including lung, colon, pancreatic, renal, and ovarian cancers. Accordingly, substances that could reduce activation could be of interest for possible treatments.

Src Homology-2 phosphatase (SHP2) is a non-receptor protein phosphatase ubiquitously expressed in various tissues and cell types (see reviews: Tajan M et al., Eur J Med Genet 2016 58(10):509-25; Grossmann K S et al., Adv Cancer Res 2010 106:53-89). SHP2 is composed of two Src homology 2 (N-SH2 and C-SH2) domains in its NH2-terminus, a catalytic PTP (protein-tyrosine phosphatase) domain, and a C-terminal tail with regulatory properties. At the basal state, the intermolecular interactions between the SH2 domains and the PTP domain prevent the access of substrates to the catalytic pocket, keeping SHP2 into a closed, auto-inhibited conformation. In response to stimulation, SHP2 activating proteins bearing phosphor-tyrosine motifs bind to the SH2 domains, leading to exposure of active site and enzymatic activation of SHP2.

### SUMMARY

ERK1/2 appear to be activated by MEK through phosphorylation of both a threonine and a tyrosine residue, namely at Tyr204/187 and Thr202/185. Once activated, ERK1/2 catalyze the phosphorylation of serine/threonine residues of more than 100 substrates and activate both cytosolic and nuclear proteins that are linked to cell growth, proliferation, survival, angiogenesis and differentiation, all hallmarks of the cancer phenotype. Thus it may be beneficial to target ERK 1 and ERK 2 to develop and use ERK1/2 inhibitors as a way to inhibit tumor growth.

Furthermore, an ERK inhibitor may have utility in combination with other kinase, for example MAPK, inhibitors. Recently, researchers reported that dual inhibition of MEK and ERK by small molecule inhibitors was synergistic and acted to overcome acquired resistance to MEK inhibitors. See Hatzivassiliou et al., ERK Inhibition Overcomes Acquired Resistance to MEK Inhibition, Mol. Cancer Ther. 2012, 11, 1143-1154.

In addition to ERK1/2, FLT3 also operates upstream of the RAS pathway.

SHP2 plays important roles in fundamental cellular functions including proliferation, differentiation, cell cycle maintenance and motility. By dephosphorylating its associated signaling molecules, SHP2 regulates multiple intracellular signaling pathways in response to a wide range of growth factors, cytokines, and hormones. Cell signaling processes in which SHP2 participates include the RAS-MAPK (mitogen-activated protein kinase), the PI3K (phosphoinositol 3-kinase)-AKT, and the JAK-STAT pathways.

SHP2 also plays a signal-enhancing role on this pathway, acting downstream of RTKs and upstream of RAS. One common mechanism of resistance for MAPK kinase inhibitors involves activation of RTKs that fuel reactivation of the MAPK signaling. RTK activation recruits SHP2 via direct binding and through adaptor proteins. Those interactions result in the conversion of SHP2 from the closed (inactive) conformation to open (active) conformation.

SHP2 is an important facilitator of RAS signaling reactivation that bypasses pharmacological inhibition in both primary and secondary resistance. Inhibition of SHP2 achieves the effect of globally attenuating upstream RTK signaling that often drives oncogenic signaling and adaptive tumor escape (see Prahallad, A. et al. Cell Reports 12, 1978-1985 (2015); Chen Y N, Nature 535, 148-152(2016)), which is incorporated herein by reference in its entirety for all of its teachings, including without limitation all methods, compounds, compositions, data, and the like, for use with any of the embodiments and disclosure herein.

The cluster of differentiation antigen 135 (CD135) also known as fins-like tyrosine kinase 3 (FLT-3), receptor-type tyrosine-protein kinase FLT3, or fetal liver kinase-2 (Flk2) is a protein that in humans is encoded by the FLT3 gene. FLT3 is a cytokine receptor belonging to the receptor tyrosine kinase class III. FLT3 is frequently mutated in acute myeloid leukemia, other hematologic malignancies, and colorectal cancer. For example, FLT3 mutations occur in about 3% of all cancers with acute myeloid leukemia, colon adenocarcinoma, lung adenocarcinoma, cutaneous melanoma, and breast invasive ductal carcinoma.

FLT3 is a receptor tyrosine kinase (RTK) which is a protein that is embedded in the cell membrane and relay growth signals from the outside environment to the cell's internal machinery. At rest, this protein resides on the cell membrane as inactive monomers. Growth factors secreted by nearby cells bind to specific RTKs, such as FL binding to FLT3, and cause these RTKs to dimerize. Dimerized RTKs activate one another through transphosphorylation of their intracellular regions. Intracellular proteins, such as adapter proteins, bind to these phosphorylated regions and propagate the pro-growth signals within the cell via one or more signaling pathways. Cells express a variety of RTKs so that environmental cues can be relayed to specific cell populations in specific contexts. FLT3 predominantly mediates pro-growth signaling in immature blood cells. Overactive RTK signaling can result in uncontrolled cell growth and survival that transforms normal cells into cancer cells.

The opportunity to target signal transduction pathways from multiple angles and potentially ameliorate feedback loops upstream of Ras via ERK1/2 or SHP2 and FLT3 provides opportunities for developing methods that employ combination therapies. The present embodiments disclosed herein generally relate to compositions and methods related to combination therapies to treat cancer utilizing an ERK1/2 inhibitor in conjunction with a FLT3 inhibitor while providing an unexpected degree synergy.

Disclosed herein is a method of treating cancer in a subject in need thereof, the method comprising: administering to the subject in need thereof a therapeutically effective amount of
(i) compound 1:

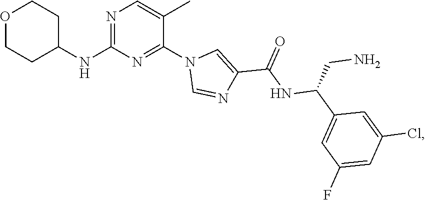 [SMILES: Cc1cnc(NC2CCOCC2)nc1-n1cnc(C(=O)N[C@H](CN)c2cc(F)cc(Cl)c2)c1]

or a pharmaceutically acceptable salt thereof; and
(ii) a FLT3 inhibitor.

Also disclosed herein is a method of treating cancer in a subject in need thereof, the method comprising: administering to the subject in need thereof a therapeutically effective amount of
(i) compound 2

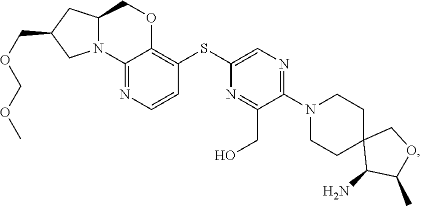 [SMILES: COCOC[C@H]1C[C@H]2COc3c(Sc4cnc(N5CCC6(CC5)CO[C@@H](C)[C@H]6N)c(CO)n4)ccnc3N2C1]

or pharmaceutically acceptable salt thereof; and
(ii) a FLT3 inhibitor.

In some embodiments, the dosing of the FLT3 inhibitor is in a range from about 1 mg/day to about 500 mg/day.

In some embodiments, the FLT3 inhibitor is crenolanib, gilteritinib, ibrutinib, lestaurtinib, midostaurin, ponatinib, quizartinib, sorafenib, sunitinib, or tandutinib.

In some embodiments, the FLT3 inhibitor is gilteritinib.

In some embodiments, gilteritinib is administered in an amount that is about 120 mg/day.

In some embodiments, gilteritinib is administered once or twice daily.

Also disclosed herein is a method of treating cancer in a subject in need thereof, the method comprising: administering to the subject in need thereof a therapeutically effective amount of
(i) compound 1:

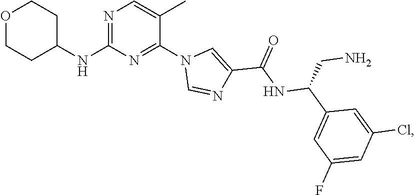 [SMILES: Cc1cnc(NC2CCOCC2)nc1-n1cnc(C(=O)N[C@H](CN)c2cc(F)cc(Cl)c2)c1]

or a pharmaceutically acceptable salt thereof; and
(ii) gilteritinib.

In some embodiments, the pharmaceutically acceptable salt of compound 1 is the mandelic acid salt.

Also disclosed herein is a method of treating cancer in a subject in need thereof, the method comprising: administering to the subject in need thereof a therapeutically effective amount of
(i) compound 2:

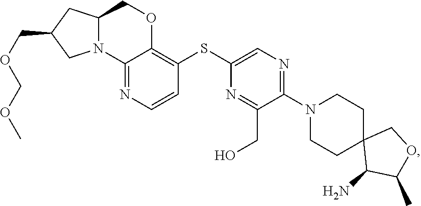 [SMILES: COCOC[C@H]1C[C@H]2COc3c(Sc4cnc(N5CCC6(CC5)CO[C@@H](C)[C@H]6N)c(CO)n4)ccnc3N2C1]

or a pharmaceutically acceptable salt thereof; and
(ii) gilteritinib.

In some embodiments, the cancer is a mitogen-activated protein kinase (MAPK) pathway driven cancer.

In some embodiments, the cancer is a BRAF-driven cancer, HRAS-driven cancer, or a NRAS-driven cancer.

In some embodiments, the cancer comprises at least one cancer cell driven by deregulated ERK.

In some embodiments, the cancer has at least one mutation in RAS. In some embodiments, the cancer has at least one mutation in RAF. In some embodiments, the cancer has at least one mutation in MEK.

In some embodiments, the cancer has a G12C KRAS mutation. In some embodiments, the cancer has a G12D KRAS mutation. In some embodiments, the cancer has a G12S KRAS mutation. In some embodiments, the cancer has a G12V KRAS mutation. In some embodiments, the cancer has a G13D KRAS mutation. In some embodiments, the cancer has a Q16H KRAS mutation. In some embodiments, the cancer has a Q16K KRAS mutation. In some embodiments, the cancer has a Q61R NRAS mutation.

In some embodiments, the cancer is a BRAF V600E or V600K mutant tumor.

In some embodiments, the cancer is a MAPKm/MAPKi-naïve pancreatic cancer.

In some embodiments, the cancer comprises one or more EGFR mutation selected from the group consisting of EGFR gene copy gain, EGFR gene amplification, chromosome 7 polysomy, L858R, exon 19 deletions/insertions, L861Q, G719C, G719S, G719A, V765A, T783A, exon 20 insertions, EGFR splice variants (Viii, Vvi, and Vii), A289D, A289T, A289V, G598A, G598V, T790M, and C797S.

In some embodiments, the cancer comprises one or more EGFR mutation selected from the group consisting of L858R, exon 19 deletion, and T790M.

In some embodiments, the cancer is a liquid tumor.

In some embodiments, the liquid tumor is leukemia.

In some embodiments, the leukemia is acute myeloid leukemia (AML). In some embodiments, the AML is relapsed and/or refractory AML. In some embodiments, the AML is a FLT3 mutant AML.

In some embodiments, the cancer is a hematologic cancer. In some embodiments, the cancer acute myeloid leukemia.

In some embodiments, the cancer is colorectal cancer.

In some embodiments, the cancer is an adenocarcinoma of the lung or colon.

In some embodiments, the cancer is cutaneous melanoma.

In some embodiments, the cancer is breast invasive ductal carcinoma.

In some embodiments, compound 1, or a pharmaceutically acceptable salt thereof, is administered in an amount that is between about 25 mg/day and about 300 mg/day.

In some embodiments, compound 1, or a pharmaceutically acceptable salt thereof, is administered in an amount that is between 25 mg/day and 150 mg/day.

In some embodiments, compound 1, or a pharmaceutically acceptable salt thereof, is administered in an amount that is about 25 mg/day, about 50 mg/day, about 75 mg/day, about 100 mg/day, about 125 mg/day, about 150 mg/day, about 175 mg/day, about 200 mg/day, about 225 mg/day, or about 250 mg/day.

In some embodiments, compound 1, or a pharmaceutically acceptable salt thereof, is administered in an amount that is about 25 mg/day, about 50 mg/day, about 100 mg/day, or about 150 mg/day.

In some embodiments, compound 2, or a pharmaceutically acceptable salt thereof, is administered in an amount that is between 20 mg/day and 400 mg/day.

In some embodiments, compound 2, or a pharmaceutically acceptable salt thereof, is administered in an amount that is between 10 mg/day and 100 mg/day.

In some embodiments, compound 2, or a pharmaceutically acceptable salt thereof, is administered in an amount that is between 20 mg/day and 80 mg/day.

In some embodiments, compound 1 or compound 2, or a pharmaceutically acceptable salt thereof, is administered once a day (QD).

In some embodiments, compound 1 or compound 2, or a pharmaceutically acceptable salt thereof, is administered twice a day (BID).

In some embodiments, compound 1 or compound 2, or a pharmaceutically acceptable salt thereof, is administered three times a day (TID).

In some embodiments, compound 1 or compound 2, or a pharmaceutically acceptable salt thereof, is administered once a week.

In some embodiments, compound 1 or compound 2, or a pharmaceutically acceptable salt thereof, is administered twice a week.

In some embodiments, compound 1, or a pharmaceutically acceptable salt thereof, is administered in an amount that is between about 25 mg and about 300 mg twice a day, once a week (BID-QW).

In some embodiments, compound 1, or a pharmaceutically acceptable salt thereof, is administered in an amount that is between about 25 mg and about 250 mg twice a day, once a week (BID-QW).

In some embodiments, compound 1, or a pharmaceutically acceptable salt thereof, is administered in an amount that is between about 25 mg and about 150 mg twice a day, once a week (BID-QW).

In some embodiments, compound 1, or a pharmaceutically acceptable salt thereof, is administered in an amount that is about 25 mg, 50 mg, about 75 mg, about 100 mg, about 125 mg, about 150 mg, about 175 mg, about 200 mg, about 225 mg, or about 250 mg twice a day, once a week (BID-QW).

In some embodiments, compound 1, or a pharmaceutically acceptable salt thereof, is administered in an amount that is about 25 mg, 50 mg, about 100 mg, about 125 mg, or about 150 mg twice a day, once a week (BID-QW).

In some embodiments, compound 1, or a pharmaceutically acceptable salt thereof, is administered in an amount that is about 125 mg twice a day, once a week (BID-QW).

In some embodiments, compound 1 or compound 2, or a pharmaceutically acceptable salt thereof, is administered for at least one 28-day cycle.

In some embodiments, compound 1 or compound 2, or a pharmaceutically acceptable salt thereof, is administered on day 1, day 8, day 15, and day 22 of a 28-day cycle.

In some embodiments, compound 1 or compound 2, or a pharmaceutically acceptable salt thereof, is administered on day 1, day 8, day 15 of a 28-day cycle.

In some embodiments, compound 1 or compound 2, or a pharmaceutically acceptable salt thereof, is administered for 2 weeks on and 1 week off (21 day schedule).

In some embodiments, compound 1 or compound 2, or a pharmaceutically acceptable salt thereof, is administered for 3 weeks on and 1 week off (28 day schedule).

In some embodiments, compound 1 or compound 2, or a pharmaceutically acceptable salt thereof, is administered three times a week (D1D3D5 TIW).

In some embodiments, compound 1 or compound 2, or a pharmaceutically acceptable salt thereof, is administered twice a day/twice a week (BID-D1D2-BIW).

In some embodiments, compound 1 or compound 2, or a pharmaceutically acceptable salt thereof, is administered orally.

In some embodiments, the dose of the FLT3 inhibitor is less than the dose required for a monotherapy with the FLT3 inhibitor.

In some embodiments, the dose of compound 1 or compound 2 is less than the dose required for a monotherapy with compound 1 or compound 2.

In some embodiments of a method of treating cancer, the method further comprises administering an additional MAPK pathway inhibitor.

In some embodiments of a method of treating cancer, the additional MAPK pathway inhibitor is a KRAS inhibitor, NRAS inhibitor, HRAS inhibitor, PDGFRA inhibitor, PDGFRB inhibitor, MET inhibitor, FGFR inhibitor, ALK inhibitor, ROS1 inhibitor, TRKA inhibitor, TRKB inhibitor, TRKC inhibitor, EGFR inhibitor, IGFR1R inhibitor, GRB2 inhibitor, SOS inhibitor, ARAF inhibitor, BRAF inhibitor, RAF1 inhibitor, MEK1 inhibitor, MEK2 inhibitor, c-Mycv, CDK4/6, inhibitor CDK2 inhibitor, FLT3 inhibitor, or ERK1/2 inhibitor.

Also disclosed herein is a kit comprising compound 1, or a pharmaceutically acceptable salt thereof, and a FLT3 inhibitor. In some embodiments of a kit, compound 1, or a pharmaceutically acceptable salt thereof, and the FLT3 inhibitor are in separate packages. In some embodiments of a kit, the kit further comprises instructions to administer the contents of the kit to a subject for the treatment of cancer. In some embodiments of a kit, the FLT inhibitor is tandutinib, ibrutinib, sorafenib, quizartinib, ponatinib, sunitinib, lestaurtinib, midostaurin, crenolanib, or gilteritinib. In some embodiments of a kit, the FLT inhibitor is gilteritinib.

Also disclosed herein is a kit comprising compound 2, or a pharmaceutically acceptable salt thereof, and a FLT3 inhibitor. In some embodiments of a kit, compound 2, or a pharmaceutically acceptable salt thereof, and the FLT3 inhibitor are in separate packages. In some embodiments of a kit, the kit further comprises instructions to administer the contents of the kit to a subject for the treatment of cancer. In some embodiments of a kit, the FLT inhibitor is tandutinib, ibrutinib, sorafenib, quizartinib, ponatinib, sunitinib, lestaurtinib, midostaurin, crenolanib, or gilteritinib. In some embodiments of a kit, the FLT inhibitor is gilteritinib.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

Various features of the present disclosure are set forth with particularity in the appended claims. A better understanding of the features and advantages of the present disclosure will be obtained by reference to the following detailed description that sets forth illustrative embodiments, in which the principles of the present disclosure are utilized, and the accompanying drawings of which:

FIG. 1A is a 3-dimensional colored map indicating that compound 2 and gilteritinib in combination showed synergy in treating FLT3-ITD mutated MOLM-14 AML cells.

FIG. 1B is a 3-dimensional colored map indicating that compound 2 and gilteritinib in combination showed synergy in treating FLT3-ITD mutated MV-4-11 AML cells.

FIG. 1C is a 3-dimensional colored map indicating that compound 2 and gilteritinib in combination showed synergy in treating FLT3-ITD mutated MOLM-13 AML cells.

FIG. 2A is a numerical grid presentation of the data graphed in FIG. 1A.

FIG. 2B is a numerical grid presentation of the data graphed in FIG. 1B.

FIG. 2C is a numerical grid presentation of the data graphed in FIG. 1C.

FIG. 3A is a Western blot showing compound 2 and gilteritinib combination induced downregulation of ERK1/2 phosphorylation in MOLM-14 cells.

FIG. 3B is a bar plot quantifying the results of FIG. 3A.

FIG. 4A is a Western blot showing compound 2 and gilteritinib combination induced downregulation of ERK1/2 phosphorylation in MV-4-11 cells.

FIG. 4B is a bar plot quantifying the results of FIG. 4A.

FIG. 5 illustrates durable tumor growth inhibition of compound 2+gilteritinib combination in vivo in FLT3-ITD mutated AML subcutaneous CDX model MOLM-13.

FIG. 6 illustrates superior tumor growth inhibition of compound 2+gilteritinib combination as compared to the respective monotherapies in vivo in FLT3-ITD mutated AML engrafted CDX model MOLM-13-Luc.

### DETAILED DESCRIPTION

As used herein and in the appended claims, the singular forms “a,” “an,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range, in some instances, will vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

As used in the specification and appended claims, unless specified to the contrary, the following terms have the meaning indicated below.

As used herein, the term “therapeutic” means an agent utilized to treat, combat, ameliorate, prevent, or improve an unwanted condition or disease of a patient. In some embodiments, a therapeutic agent such as a compound 1 is directed to the treatment and/or the amelioration of cancers.

“Administering” when used in conjunction with a therapeutic means to administer a therapeutic systemically or locally, as directly into or onto a target tissue, or to administer a therapeutic to a patient whereby the therapeutic positively impacts the tissue to which it is targeted. Thus, as used herein, the term “administering,” when used in conjunction with a composition described herein, can include, but is not limited to, providing a composition into or onto the target tissue; providing a composition systemically to a patient by, e.g., oral administration whereby the therapeutic reaches the target tissue or cells. “Administering” a composition may be accomplished by injection, topical administration, and oral administration or by other methods alone or in combination with other known techniques.

The term “animal” as used herein includes, but is not limited to, humans and non-human vertebrates such as wild, domestic and farm animals. As used herein, the terms “patient,” “subject” and “individual” are intended to include living organisms in which certain conditions as described herein can occur. Examples include humans, monkeys, cows, sheep, goats, dogs, cats, mice, rats, and transgenic species thereof. In a preferred embodiment, the patient is a primate. In certain embodiments, the primate or subject is a human. In certain instances, the human is an adult. In certain instances, the human is child. In further instances, the human is under the age of 12 years. In certain instances, the human is elderly. In other instances, the human is 60 years of age or older. Other examples of subjects include experimental animals such as mice, rats, dogs, cats, goats, sheep, pigs, and cows. The experimental animal can be an animal model for a disorder, e.g., a transgenic mouse with hypertensive pathology.

By “pharmaceutically acceptable,” it is meant the carrier, diluent or excipient must be compatible with the other ingredients of the formulation and not deleterious to the recipient thereof.

The term “pharmaceutical composition” shall mean a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

A “therapeutically effective amount” or “effective amount” as used herein refers to the amount of active compound or pharmaceutical agent that elicits a biological or medicinal response in a tissue, system, animal, individual or human that is being sought by a researcher, veterinarian, medical doctor or other clinician, which includes one or more of the following: (1) preventing the disease; for example, preventing a disease, condition or disorder in an individual that may be predisposed to the disease, condition or disorder but does not yet experience or display the pathology or symptomatology of the disease, (2) inhibiting the disease; for example, inhibiting a disease, condition or disorder in an individual that is experiencing or displaying the pathology or symptomatology of the disease, condition or disorder (i.e., arresting further development of the pathology and/or symptomatology), and (3) ameliorating the disease; for example, ameliorating a disease, condition or disorder in an individual that is experiencing or displaying the pathology or symptomatology of the disease, condition or disorder (i.e., reversing the pathology and/or symptomatology).

The terms “treat,” “treated,” “treatment,” or “treating” as used herein refers to both therapeutic treatment in some embodiments and prophylactic or preventative measures in other embodiments, wherein the object is to prevent or slow (lessen) an undesired physiological condition, disorder, or disease, or to obtain beneficial or desired clinical results. For the purposes described herein, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment. A prophylactic benefit of treatment includes prevention of a condition, retarding the progress of a condition, stabilization of a condition, or decreasing the likelihood of occurrence of a condition. As used herein, “treat,” “treated,” “treatment,” or“treating” includes prophylaxis in some embodiments.

The term “substantially the same as” as used herein, refers to a powder x-ray diffraction pattern or differential scanning calorimetry pattern that is non-identical to those depicted herein, but that falls within the limits of experimental error, when considered by one of ordinary skill in the art.

“Cancer” refers to all types of cancer, neoplasm or malignant tumors found in mammals (e.g. humans), including, without limitation, leukemias, lymphomas, myelomas, carcinomas, and sarcomas. Exemplary cancers that may be treated with a compound or method provided herein include brain cancer, glioma, glioblastoma, neuroblastoma, prostate cancer, colorectal cancer, pancreatic cancer (such as pancreatic adenocarcinoma, PDAC), medulloblastoma, melanoma, cervical cancer, gastric cancer, ovarian cancer, lung cancer, cancer of the head, Hodgkin's Disease, and Non-Hodgkin's Lymphomas. Exemplary cancers that may be treated with a compound or method provided herein include cancer of the blood, thyroid, endocrine system, brain, breast, cervix, colon, head & neck, liver, kidney, lung, ovary, pancreas, rectum, stomach, and uterus. Additional examples include, thyroid carcinoma, cholangiocarcinoma, pancreatic adenocarcinoma, skin cutaneous melanoma, colon adenocarcinoma, rectum adenocarcinoma, stomach adenocarcinoma, esophageal carcinoma, head and neck squamous cell carcinoma, breast invasive carcinoma, lung adenocarcinoma, lung squamous cell carcinoma, non-small cell lung carcinoma, mesothelioma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer. In some embodiments the cancer is colorectal cancer (CRC), pancreatic ductal adenocarcinoma (PDAC), cholangiocarcinoma cancer, appendiceal cancer, gastric cancer, esophageal cancer, non-small cell lung cancer (NSCLC), head and neck cancer, ovarian cancer, uterine cancer, acute myeloid leukemia (AML), or melanoma.

“FLT3” refers to Fms Related Receptor Tyrosine Kinase, also known as CD135 or CD135 antigen, Fms-Like Tyrosine Kinase, FLK2, STK1 Stem Cell Tyrosine Kinase 1 Fms Related Tyrosine Kinase 3, or FL Cytokine Receptor 3 and is a class III receptor tyrosine kinase that regulates hematopoiesis. This receptor is activated by binding of the fins-related tyrosine kinase 3 ligand to the extracellular domain, which induces homodimer formation in the plasma membrane leading to autophosphorylation of the receptor. The activated receptor kinase phosphorylates and activates multiple cytoplasmic effector molecules in pathways involved in apoptosis, proliferation, and differentiation of hematopoietic cells. Mutations that result in the constitutive activation of this receptor result in acute myeloid leukemia and acute lymphoblastic leukemia.

“Subject” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, horse, and other non-mammalian animals. In some embodiments, the patient is human.

### Compound 1

Disclosed herein is (S)-N-(2-amino-1-(3-chloro-5-fluorophenyl)ethyl)-1-(5-methyl-2-((tetrahydro-2H-pyran-4-yl)amino)pyrimidin-4-yl)-1H-imidazole-4-carboxamide:

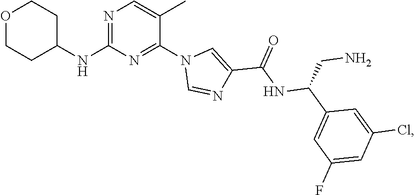 [SMILES: Cc1cnc(NC2CCOCC2)nc1-n1cnc(C(=O)N[C@H](CN)c2cc(F)cc(Cl)c2)c1]

or a pharmaceutically acceptable salt thereof.

In some embodiments, the salt of compound 1 is the mandelic acid salt. In some embodiments, the salt of compound 1 is the benzenesulfonic acid salt. In some embodiments, the salt of compound 1 is the hydrochloride salt. In some embodiments, the salt of compound 1 is the p-toluenesulfonic acid salt.

In some embodiments, the salt of compound 1 is the benzenesulfonic acid salt.

### Compound 2

Disclosed herein is (3-((3S,4S)-4-amino-3-methyl-2-oxa-8-azaspiro[4.5]decan-8-yl)-6-(((6aS,8S)-8-((methoxymethoxy)methyl)-6a,7,8,9-tetrahydro-6H-pyrido[3,2-b]pyrrolo[1,2-d][1,4]oxazin-4-yl)thio)pyrazin-2-yl)methanol:

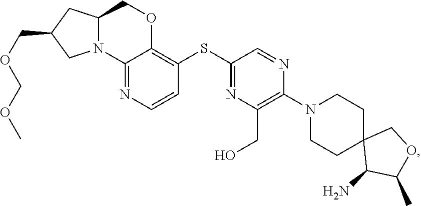 [SMILES: COCOC[C@H]1C[C@H]2COc3c(Sc4cnc(N5CCC6(CC5)CO[C@@H](C)[C@H]6N)c(CO)n4)ccnc3N2C1]

or a pharmaceutically acceptable salt thereof.

### FL T3 Inhibitors

FLT 3 inhibitors are tyrosine kinase inhibitors that compete for the ATP binding site in the active domain of the kinase, and thus prevent phosphorylation of the protein and decrease the protein's activity.

Type I inhibitors bind to the ATP-binding site when the receptor is active, while type II inhibitors interact with a hydrophobic region immediately adjacent to the ATP-binding site, which is accessible when the receptor is in its inactive conformation. Type I inhibitors include sunitinib, lestaurtinib, midostaurin, crenolanib and gilteritinib, while type II inhibitors include sorafenib, quizartinib and ponatinib.

In some embodiments, the FLT3 inhibitor is crenolanib, gilteritinib, ibrutinib, lestaurtinib, midostaurin, ponatinib, quizartinib, sorafenib, sunitinib, or tandutinib. In some embodiments, the FLT3 inhibitor is crenolanib. In some embodiments, the FLT3 inhibitor is gilteritinib. Gilteritinib is sold under the brand name Xospata™ by Astellas Pharma US, Inc. In some embodiments, the FLT3 inhibitor is ibrutinib. In some embodiments, the FLT3 inhibitor is lestaurtinib. In some embodiments, the FLT3 inhibitor is midostaurin. In some embodiments, the FLT3 inhibitor is ponatinib. In some embodiments, the FLT3 inhibitor is quizartinib. In some embodiments, the FLT3 inhibitor is sorafenib. In some embodiments, the FL73 inhibitor is sunitinib. In some embodiments, the FLT3 inhibitor is tandutinib.

In some embodiments, the FLT3 inhibitor is ibrutinib, ponatinib, quizartinib, crenolanib, or gilteritinib. In some embodiments, the FLT3 inhibitor is sorafenib, lestaurtinib, midostaurin, sunitinib, or tandutinib. In some embodiments, a combination using two or more of the FLT3 inhibitor enumerated above can be combined, such as combining a type I FLT3 inhibitor with a type II FLT3 inhibitor. As an example, gilteritinib can be combined with quizartinib. In some embodiments, one or more of the inhibitors listed above and elsewhere herein can be specifically excluded from the embodiments set forth herein, including without limitation, any methods, kits, and compositions of matter.

### Further Forms

The compound disclosed herein may exist as salts. The present embodiments includes such salts, which can be pharmaceutically acceptable salts. Examples of applicable salt forms include hydrochlorides, hydrobromides, sulfates, methanesulfonates, nitrates, maleates, acetates, citrates, fumarates, tartrates (e.g., (+)-tartrates, (−)-tartrates or mixtures thereof including racemic mixtures, succinates, benzoates, and salts with amino acids such as glutamic acid. These salts may be prepared by methods known to those skilled in art. Also included are base addition salts such as sodium, potassium, calcium, ammonium, organic amino, or magnesium salt, or a similar salt. When compounds of the present embodiments contain relatively basic functionalities, acid addition salts can be obtained by contacting the neutral form of such compounds with a sufficient amount of the desired acid, either neat or in a suitable inert solvent. Examples of acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like. Certain specific compounds of the present embodiments contain both basic and acidic functionalities that allow the compounds to be converted into either base or acid addition salts.

Other salts include acid or base salts of the compounds used in the methods of the present embodiments. Illustrative examples of pharmaceutically acceptable salts are mineral acid (hydrochloric acid, hydrobromic acid, phosphoric acid, and the like) salts, organic acid (acetic acid, propionic acid, glutamic acid, citric acid, and the like) salts, and quaternary ammonium(methyl iodide, ethyl iodide, and the like) salts. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference n its entirety for all of its teachings, including without limitation all methods, compounds, compositions, data, and the like, for use with any of the embodiments and disclosure herein.

Pharmaceutically acceptable salts includes salts of the active compounds which are prepared with relatively nontoxic acids or bases, depending on the particular substituents found on the compounds described herein. When compounds of the present embodiments contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present embodiments contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19), which is incorporated herein by reference in its entirety for all of its teachings, including without limitation all methods, compounds, compositions, data, and the like, for use with any of the embodiments and disclosure herein. Certain specific compounds of the present embodiments contain both basic and acidic functionalities that allow the compounds to be converted into either base or acid addition salts.

The neutral forms of the compounds are preferably regenerated by contacting the salt with a base or acid and isolating the parent compound in the conventional manner. The parent form of the compound differs from the various salt forms in certain physical properties, such as solubility in polar solvents.

Certain compounds of the present embodiments can exist in unsolvated forms as well as solvated forms, including hydrated forms. In general, the solvated forms are equivalent to unsolvated forms and are encompassed within the scope of the present embodiments. Certain compounds of the present embodiments may exist in multiple crystalline or amorphous forms. In general, all physical forms are equivalent for the uses contemplated by the present embodiments and are intended to be within the scope of the present embodiments.

Certain compounds of the present embodiments possess asymmetric carbon atoms (optical centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present embodiments. The compounds of the present embodiments do not include those that are known in art to be too unstable to synthesize and/or isolate. The present embodiments is meant to include compounds in racemic and optically pure forms. Optically active (R)- and (S)-, or (D)- and (L)-isomers may be prepared using chiral synthons or chiral reagents, or resolved using conventional techniques.

Unless otherwise stated, the compounds of the present embodiments may also contain unnatural proportions of atomic isotopes at one or more of the atoms that constitute such compounds. For example, the compounds of the present embodiments may be labeled with radioactive or stable isotopes, such as for example deuterium (²H), tritium (³H), iodine-125 (¹²⁵I), fluorine-18 (¹⁸F), nitrogen-15 (¹⁵N), oxygen-17 (¹⁷O), oxygen-18 (¹⁸O), carbon-13 (¹³C), or carbon-14 (⁴C). All isotopic variations of the compounds of the present embodiments, whether radioactive or not, are encompassed within the scope of the present embodiments.

In addition to salt forms, the present embodiments provide compounds, which are in a prodrug form. Prodrugs of the compounds described herein are those compounds that readily undergo chemical changes under physiological conditions to provide the compounds of the present embodiments. Additionally, prodrugs can be converted to the compounds of the present embodiments by chemical or biochemical methods in an ex vivo environment. For example, prodrugs can be slowly converted to the compounds of the present embodiments when placed in a transdermal patch reservoir with a suitable enzyme or chemical reagent.

### Combinations

Disclosed herein is a method of treating cancer in a subject in need thereof, the method comprising: administering to the subject in need thereof a therapeutically effective amount of
(i) compound 1:

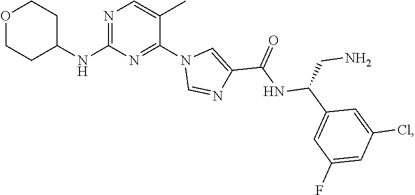 [SMILES: Cc1cnc(NC2CCOCC2)nc1-n1cnc(C(=O)N[C@H](CN)c2cc(F)cc(Cl)c2)c1]

or a pharmaceutically acceptable salt thereof, and
(ii) a FLT3 inhibitor.

Disclosed herein is a method of treating cancer in a subject in need thereof, the method comprising: administering to the subject in need thereof a therapeutically effective amount of
(i) compound 1:

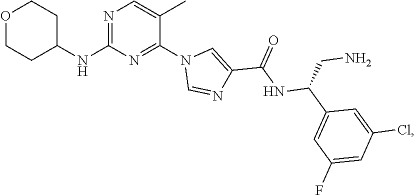 [SMILES: Cc1cnc(NC2CCOCC2)nc1-n1cnc(C(=O)N[C@H](CN)c2cc(F)cc(Cl)c2)c1]

or a pharmaceutically acceptable salt thereof, and
(ii) gilteritinib.

Disclosed herein is a method of treating a hematologic cancer in a subject in need thereof, the method comprising: administering to the subject in need therapeutically effective amount of
(i) compound 1:

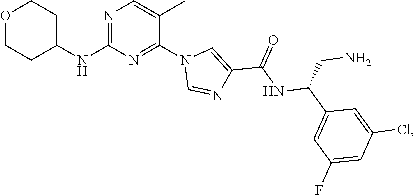 [SMILES: Cc1cnc(NC2CCOCC2)nc1-n1cnc(C(=O)N[C@H](CN)c2cc(F)cc(Cl)c2)c1]

or a pharmaceutically acceptable salt thereof, and
(ii) gilteritinib.

Disclosed herein is a method of treating acute myeloid leukemia in a subject in need thereof, the method comprising: administering to the subject in need thereof a therapeutically effective amount of
(i) compound 1:

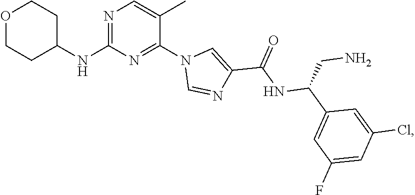 [SMILES: Cc1cnc(NC2CCOCC2)nc1-n1cnc(C(=O)N[C@H](CN)c2cc(F)cc(Cl)c2)c1]

or a pharmaceutically acceptable salt thereof, and
(ii) gilteritinib.

Disclosed herein is a method of treating colorectal cancer in a subject in need thereof, the method comprising: administering to the subject in need thereof a therapeutically effective amount of
(i) compound 1:

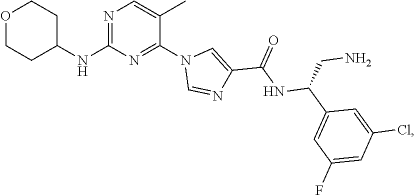 [SMILES: Cc1cnc(NC2CCOCC2)nc1-n1cnc(C(=O)N[C@H](CN)c2cc(F)cc(Cl)c2)c1]

or a pharmaceutically acceptable salt thereof, and
(ii) gilteritinib.

Disclosed herein is a method of treating colon or lung cancer in a subject in need thereof, the method comprising: administering to the subject in need thereof a therapeutically effective amount of
(i) compound 1:

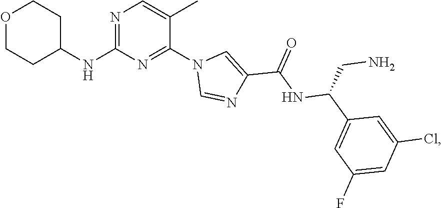 [SMILES: Cc1cnc(NC2CCOCC2)nc1-n1cnc(C(=O)N[C@H](CN)c2cc(F)cc(Cl)c2)c1]

or a pharmaceutically acceptable salt thereof, and
(ii) gilteritinib.

In some embodiments, the colon or lung cancer is colon or lung adenocarcinoma.

Disclosed herein is a method of treating melanoma in a subject in need thereof, the method comprising: administering to the subject in need thereof a therapeutically effective amount of
(i) compound 1:

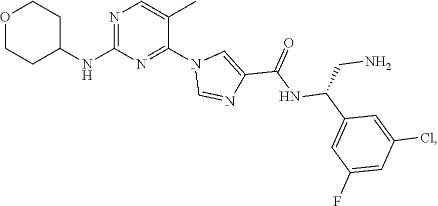 [SMILES: Cc1cnc(NC2CCOCC2)nc1-n1cnc(C(=O)N[C@H](CN)c2cc(F)cc(Cl)c2)c1]

or a pharmaceutically acceptable salt thereof, and
(ii) gilteritinib.

In some embodiments, the melanoma is cutaneous melanoma.

Disclosed herein is a method of treating breast cancer in a subject in need thereof, the method comprising: administering to the subject in need thereof a therapeutically effective amount of
(i) compound 1:

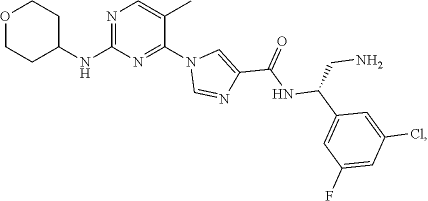 [SMILES: Cc1cnc(NC2CCOCC2)nc1-n1cnc(C(=O)N[C@H](CN)c2cc(F)cc(Cl)c2)c1]

or a pharmaceutically acceptable salt thereof, and
(ii) gilteritinib.

In some embodiments, the breast cancer is breast invasive ductal carcinoma.

Disclosed herein is a method of treating cancer in a subject in need thereof, the method comprising: administering to the subject in need thereof a therapeutically effective amount of
(i) compound 2:

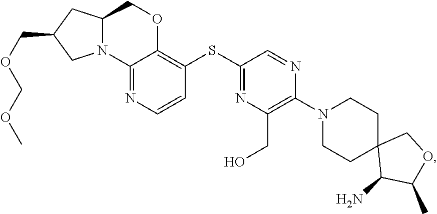 [SMILES: COCOC[C@H]1C[C@H]2COc3c(Sc4cnc(N5CCC6(CC5)CO[C@@H](C)[C@H]6N)c(CO)n4)ccnc3N2C1]

or a pharmaceutically acceptable salt thereof, and
(ii) a FLT3 inhibitor.

Disclosed herein is a method of treating cancer in a subject in need thereof, the method comprising: administering to the subject in need thereof a therapeutically effective amount of
(i) compound 2:

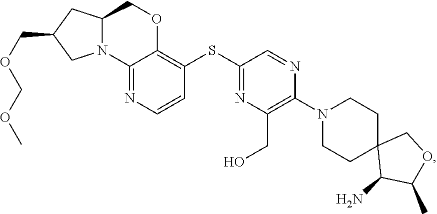 [SMILES: COCOC[C@H]1C[C@H]2COc3c(Sc4cnc(N5CCC6(CC5)CO[C@@H](C)[C@H]6N)c(CO)n4)ccnc3N2C1]

or a pharmaceutically acceptable salt thereof, and
(ii) gilteritinib.

Disclosed herein is a method of treating a hematologic cancer in a subject in need thereof, the method comprising: administering to the subject in need thereof a therapeutically effective amount of
(i) compound 2:

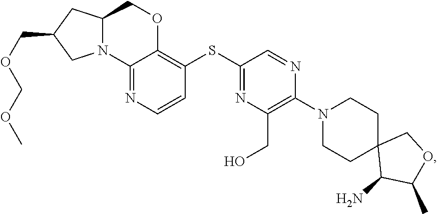 [SMILES: COCOC[C@H]1C[C@H]2COc3c(Sc4cnc(N5CCC6(CC5)CO[C@@H](C)[C@H]6N)c(CO)n4)ccnc3N2C1]

or a pharmaceutically acceptable salt thereof, and
(ii) gilteritinib.

Disclosed herein is a method of treating acute myeloid leukemia in a subject in need thereof, the method comprising: administering to the subject in need thereof a therapeutically effective amount of
(i) compound 2:

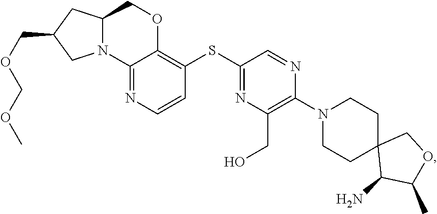 [SMILES: COCOC[C@H]1C[C@H]2COc3c(Sc4cnc(N5CCC6(CC5)CO[C@@H](C)[C@H]6N)c(CO)n4)ccnc3N2C1]

or a pharmaceutically acceptable salt thereof, and
(ii) gilteritinib.

Disclosed herein is a method of treating colorectal cancer in a subject in need thereof, the method comprising: administering to the subject in need thereof a therapeutically effective amount of
(i) compound 2:

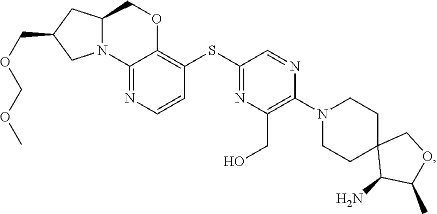 [SMILES: COCOC[C@H]1C[C@H]2COc3c(Sc4cnc(N5CCC6(CC5)CO[C@@H](C)[C@H]6N)c(CO)n4)ccnc3N2C1]

or a pharmaceutically acceptable salt thereof, and
(ii) gilteritinib.

Disclosed herein is a method of treating colon or lung cancer in a subject in need thereof, the method comprising: administering to the subject in need thereof a therapeutically effective amount of
(i) compound 2:

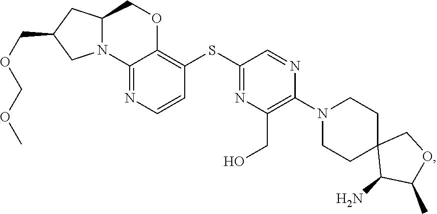 [SMILES: COCOC[C@H]1C[C@H]2COc3c(Sc4cnc(N5CCC6(CC5)CO[C@@H](C)[C@H]6N)c(CO)n4)ccnc3N2C1]

or a pharmaceutically acceptable salt thereof, and
(ii) gilteritinib.

In some embodiments, the colon or lung cancer is colon or lung adenocarcinoma.

Disclosed herein is a method of treating melanoma in a subject in need thereof, the method comprising: administering to the subject in need thereof a therapeutically effective amount of
(i) compound 2:

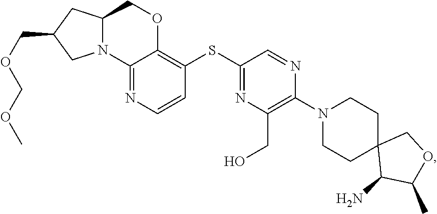 [SMILES: COCOC[C@H]1C[C@H]2COc3c(Sc4cnc(N5CCC6(CC5)CO[C@@H](C)[C@H]6N)c(CO)n4)ccnc3N2C1]

or a pharmaceutically acceptable salt thereof, and
(ii) gilteritinib.

In some embodiments, the melanoma is cutaneous melanoma.

Disclosed herein is a method of treating breast cancer in a subject in need thereof, the method comprising: administering to the subject in need thereof a therapeutically effective amount of
(i) compound 2:

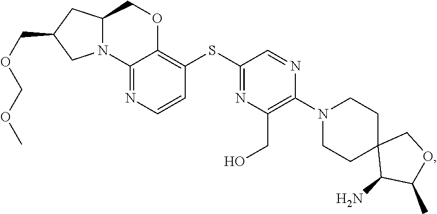 [SMILES: COCOC[C@H]1C[C@H]2COc3c(Sc4cnc(N5CCC6(CC5)CO[C@@H](C)[C@H]6N)c(CO)n4)ccnc3N2C1]

or a pharmaceutically acceptable salt thereof, and
(ii) gilteritinib.

In some embodiments, the breast cancer is breast invasive ductal carcinoma.

### Further Combination

In some embodiments, the method comprises administering an additional MAPK pathway inhibitor. Without being bound by theory, suppression of MAPK signaling in cancer cells can result in downregulation of PD-L1 expression and increase the likelihood that the cancer cells are detected by the immune system. Such third MAPK pathway inhibitors may be based on other mutations of proteins in the MAPK pathway. In some embodiments, the additional MAPK pathway inhibitor inhibits a protein in the MAPK pathway. In some embodiments, the additional MAPK pathway inhibitor inhibits a protein outside the MAPK pathway. In some embodiments, the additional MAPK pathway inhibitor is a KRAS inhibitor, NRAS inhibitor, HRAS inhibitor, PDGFRA inhibitor, PDGFRB inhibitor, MET inhibitor, FGFR inhibitor, ALK inhibitor, ROS1 inhibitor, TRKA inhibitor, TRKB inhibitor, TRKC inhibitor, EGFR inhibitor, IGFR1R inhibitor, GRB2 inhibitor, SOS inhibitor, ARAF inhibitor, BRAF inhibitor, RAF1 inhibitor, MEK1 inhibitor, MEK2 inhibitor, c-Mycv, CDK4/6, inhibitor CDK2 inhibitor, FLT3 inhibitor, or ERK1/2 inhibitor. Exemplary MAPK pathway inhibitors include, without limitation, adagrasib, afatinib, ASTX029, binimetinib, cetuximab, cobimetinib, dabrafenib, dacomitinib, encorafenib, erlotinib, gefitinib, lapatinib, LTT462, LY3214996, necitumumab, neratinib, nimotuzumab, osimertinib, palbociclib, panitumumab, selumetinib, sotorasib, trametinib, ulixertinib, and vandetanib.

In some embodiment the additional MAPK pathway inhibitor is adagrasib. In some embodiment the additional MAPK pathway inhibitor is afatinib. In some embodiment the additional MAPK pathway inhibitors is binimetinib. In some embodiment the additional MAPK pathway inhibitor is cetuximab. In some embodiment the additional MAPK pathway inhibitor is cobimetinib. In some embodiment the additional MAPK pathway inhibitor is dabrafenib. In some embodiment the additional MAPK pathway inhibitor is dacomitinib. In some embodiment the additional MAPK pathway inhibitor is encorafenib. In some embodiment the additional MAPK pathway inhibitor is erlotinib. In some embodiment the additional MAPK pathway inhibitor is gefitinib. In some embodiment the additional MAPK pathway inhibitor is lapatinib. In some embodiment the additional MAPK pathway inhibitor is LTT462. In some embodiment the additional MAPK pathway inhibitor is LY3214996. In some embodiment the additional MAPK pathway inhibitor is necitumumab. In some embodiment the additional MAPK pathway inhibitor is neratinib. In some embodiment the additional MAPK pathway inhibitor is nimotuzumab. In some embodiment the additional MAPK pathway inhibitor is osimertinib. In some embodiment the additional MAPK pathway inhibitor is palbociclib. In some embodiment the additional MAPK pathway inhibitor is panitumumab. In some embodiment the additional MAPK pathway inhibitor is selumetinib. In some embodiment the additional MAPK pathway inhibitor is sotorasib. In some embodiment the additional MAPK pathway inhibitor is trametinib. In some embodiment the additional MAPK pathway inhibitor is ulixertinib. In some embodiment the additional MAPK pathway inhibitor is vandetanib.

The methods disclosed herein can be combined with other chemotherapeutic agents. Examples of such agents can be found in Cancer Principles and Practice of Oncology by V. T. Devita and S. Hellman (editors), 6th edition (Feb. 15, 2001), Lippincott Williams & Wilkins Publishers; which is incorporated herein by reference in its entirety for all of its teachings, including without limitation all methods, compounds, compositions, data, and the like, for use with any of the embodiments and disclosure herein. A person of ordinary skill in the art would be able to discern which combinations of agents would be useful based on the particular characteristics of the drugs and the disease involved.

In embodiments, the methods can include the co-administration of at least one cytotoxic agent. The term “cytotoxic agent” as used herein refers to a substance that inhibits or prevents a cellular function and/or causes cell death or destruction. Cytotoxic agents include, but are not limited to, radioactive isotopes (e.g., At211, I131, 1125, Y90, Re186, Re188, Sm153, Bi212, P32, Pb212 and radioactive isotopes of Lu); chemotherapeutic agents; growth inhibitory agents; enzymes and fragments thereof such as nucleolytic enzymes; and toxins such as small molecule toxins or enzymatically active toxins of bacterial, fungal, plant or animal origin, including fragments and/or variants thereof.

Examples of cytotoxic agents can be selected from anti-microtubule agents, platinum coordination complexes, alkylating agents, antibiotic agents, topoisomerase II inhibitors, antimetabolites, topoisomerase I inhibitors, hormones and hormonal analogues, signal transduction pathway inhibitors, non-receptor tyrosine kinase angiogenesis inhibitors, immunotherapeutic agents, proapoptotic agents, inhibitors of LDH-A; inhibitors of fatty acid biosynthesis; cell cycle signaling inhibitors; HDAC inhibitors, proteasome inhibitors; and inhibitors of cancer metabolism.

Chemotherapeutic agents include chemical compounds useful in the treatment of cancer. Examples of chemotherapeutic agents include erlotinib (TARCEVA®, Genentech/OSI Pharm.), bortezomib (VELCADE®, Millennium Pharm.), disulfiram, epigallocatechin gallate, salinosporamide A, carfilzomib, 17-AAG(geldanamycin), radicicol, lactate dehydrogenase A (LDH-A), fulvestrant, sunitinib, letrozole, imatinib mesylate, finasunate, oxaliplatin, 5-FU (5-fluorouracil), leucovorin, Rapamycin, Lapatinib, Lonafarnib, sorafenib, gefitinib, AG1478, alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including topotecan and irinotecan); bryostatin; callystatin; CC 1065 (including its adozelesin, carzelesin and bizelesin synthetic analogs); cryptophycins (particularly cryptophycin 1 and cryptophycin 8); adrenocorticosteroids (including prednisone and prednisolone); cyproterone acetate; Salpha-reductases including finasteride and dutasteride); vorinostat, romidepsin, panobinostat, valproic acid, mocetinostat dolastatin; aldesleukin, talc duocarmycin (including the synthetic analogs, KW-2189 and CB1-TM1); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin γ1I and calicheamicin ω1I (Angew Chem. Intl. Ed. Engl. 1994 33:183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin, morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6 azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidamnol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel, ABRAXANE® (Cremophor-free), albumin-engineered nanoparticle formulations of paclitaxel (American Pharmaceutical Partners, Schaumberg, Ill.), and docetaxel, chloranmbucil; gemcitabine; 6-thioguanine; mercaptopurine; methotrexate; platinum analogs such as cisplatin and carboplatin; vinblastine; etoposide (VP-16); ifosfamide; mitoxantrone; vincristine; vinorelbine; novantrone; teniposide; edatrexate; daunomycin; aminopterin; capecitabine; ibandronate; CPT-11; topoisomerase inhibitor RFS 2000; difluoromethylornithine (DMFO); retinoids such as retinoic acid; and pharmaceutically acceptable salts, acids and derivatives of any of the above.

Chemotherapeutic agent also includes (i) anti-hormonal agents that act to regulate or inhibit hormone action on tumors such as anti-estrogens and selective estrogen receptor modulators (SERMs), including, for example, tamoxifen, raloxifene, droloxifene, iodoxyfene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4(5)-imidazoles, aminoglutethimide, megestrol acetate, exemestane, formestanie, fadrozole, vorozole, letrozole, and anastrozole; (iii) anti-androgens such as flutamide, nilutamide, bicalutamide, leuprolide and goserelin; buserelin, tripterelin, medroxyprogesterone acetate, diethylstilbestrol, premarin, fluoxymesterone, all transretionic acid, fenretinide, as well as troxacitabine (a 1,3-dioxolane nucleoside cytosine analog); (iv) protein kinase inhibitors; (v) lipid kinase inhibitors; (vi) antisense oligonucleotides, particularly those which inhibit expression of genes in signaling pathways implicated in aberrant cell proliferation, such as, for example, PKC-alpha, Ralf and H-Ras; (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; PROLEUKIN®, rIL-2; a topoisomerase 1 inhibitor such as LURTOTECAN®; ABARELIX® rmRH; and (ix) pharmaceutically acceptable salts, acids and derivatives of any of the above.

Chemotherapeutic agent also includes antibodies such as alemtuzumab (Campath), bevacizumab; cetuximab; panitumumab, rituximab, pertuzumab, trastuzumab, tositumomab, and the antibody drug conjugate, gemtuzumab ozogamicin. Additional humanized monoclonal antibodies with therapeutic potential as agents in combination with the compounds of the present disclosure include: apolizumab, aselizumab, atlizumab, bapineuzumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, tucotuzumab celmoleukin, tucusituzumab, umavizumab, urtoxazumab, ustekinumab, visilizumab, and the anti-interleukin-12 (ABT-874/J695, Wyeth Research and Abbott Laboratories) which is a recombinant exclusively human-sequence, full-length IgGI λ antibody genetically modified to recognize interleukin-12 p40 protein.

Chemotherapeutic agent also includes “EGFR inhibitors,” which refers to compounds that bind to or otherwise interact directly with EGFR or its mutant forms and prevent or reduce its signaling activity, and is alternatively referred to as an “EGFR antagonist.” Examples of such agents include antibodies and small molecules that bind to EGFR. Examples of antibodies which bind to EGFR include MAb 579 (ATCC CRL HB 8506), MAb 455 (ATCC CRL HB8507), MAb 225 (ATCC CRL 8508), MAb 528 (ATCC CRL 8509) (see, U.S. Pat. No. 4,943,533, Mendelsohn et al.) and variants thereof, such as chimerized 225 (C225 or Cetuximab) and reshaped human 225 (H225) (see, WO 96/40210, Imclone Systems Inc.); IMC-11F8, a fully human, EGFR-targeted antibody (Imclone); antibodies that bind type II mutant EGFR (U.S. Pat. No. 5,212,290); humanized and chimeric antibodies that bind EGFR as described in U.S. Pat. No. 5,891,996; and human antibodies that bind EGFR, such as ABX-EGF or Panitumumab (see WO98/50433, Abgenix/Amgen); EMD 55900 (Stragliotto et al. Eur. J. Cancer 32A: 636-640 (1996)); EMD7200 (matuzumab) a humanized EGFR antibody directed against EGFR that competes with both EGF and TGF-alpha for EGFR binding (EMD/Merck); human EGFR antibody, HuMax-EGFR (GenMab); fully human antibodies known as E1.1, E2.4, E2.5, E6.2, E6.4, E2.11, E6.3 and E7.6.3 and described in U.S. Pat. No. 6,235,883; MDX-447 (Medarex Inc); and mAb 806 or humanized mAb 806 (Johns et al., J. Biol. Chem. 279(29):30375-30384 (2004)). The anti-EGFR antibody may be conjugated with a cytotoxic agent, thus generating an immunoconjugate (see, e.g., EP659,439A2, Merck Patent GmbH). EGFR antagonists include small molecules such as compounds described in U.S. Pat. Nos. 5,616,582, 5,457,105, 5,475,001, 5,654,307, 5,679,683, 6,084,095, 6,265,410, 6,455,534, 6,521,620, 6,596,726, 6,713,484, 5,770,599, 6,140,332, 5,866,572, 6,399,602, 6,344,459, 6,602,863, 6,391,874, 6,344,455, 5,760,041, 6,002,008, and 5,747,498, as well as the following PCT publications: WO98/14451, WO98/50038, WO99/09016, and WO99/24037. Particular small molecule EGFR antagonists include OSI-774 (CP-358774, erlotinib, TARCEVA® Genentech/OSI Pharmaceuticals); PD 183805 (CI 1033, 2-propenamide, N-[4-[(3-chloro-4-fluorophenyl)amino]-7-[3-(4-morpholinyl)propoxy]-6-quinazolinyl]-, dihydrochloride, Pfizer Inc.); ZD1839, gefitinib, 4-(3′-Chloro-4′-fluoroanilino)-7-methoxy-6-(3-morpholinopropoxy)quinazoline, AstraZeneca); ZM 105180 ((6-amino-4-(3-methylphenyl-amino)-quinazoline, Zeneca); BIBX-1382 (N8-(3-chloro-4-fluoro-phenyl)-N2-(1-methyl-piperidin-4-yl)-pyrimido[5,4-d]pyrimidine-2,8-diamine, Boehringer Ingelheim); PKI-166 ((R)-4-[4-[(1-phenylethyl)amino]-1H-pyrrolo[2,3-d]pyrimidin-6-yl]-phenol); (R)-6-(4-hydroxyphenyl)-4-[(1-phenylethyl)amino]-7H-pyrrolo[2,3-d]pyrimidine); CL-387785 (N-[4-[(3-bromophenyl)amino]-6-quinazolinyl]-2-butynamide); EKB-569 (N-[4-[(3-chloro-4-fluorophenyl)amino]-3-cyano-7-ethoxy-6-quinolinyl]-4-(dimethylamino)-2-butenamide) (Wyeth); AG1478 (Pfizer); AG1571 (SU 5271; Pfizer); dual EGFR/HER2 tyrosine kinase inhibitors such as lapatinib (TYKERB®, GSK572016 or N-[3-chloro-4-[(3 fluorophenyl)methoxy]phenyl]-6[5[[[2methylsulfonyl)ethyl]amino]methyl]-2-furanyl]-4-quinazolinamine). Each of the above-described references is incorporated herein by reference in its entirety for all of its teachings, including without limitation all methods, compounds, compositions, data, and the like, for use with any of the embodiments and disclosure herein.

Chemotherapeutic agents also include “tyrosine kinase inhibitors” including the EGFR-targeted drugs noted in the preceding paragraph; small molecule HER2 tyrosine kinase inhibitor such as TAK165 available from Takeda; CP-724,714, an oral selective inhibitor of the ErbB2 receptor tyrosine kinase (Pfizer and OSI); dual-HER inhibitors such as EKB-569 (available from Wyeth) which preferentially binds EGFR but inhibits both HER2 and EGFR-overexpressing cells; lapatinib (GSK572016; available from Glaxo-SmithKline), an oral HER2 and EGFR tyrosine kinase inhibitor; PKI-166 (available from Novartis); pan-HER inhibitors such as canertinib (CI-1033; Pharmacia); Raf-1 inhibitors such as antisense agent ISIS-5132 available from ISIS Pharmaceuticals which inhibit Raf-1 signaling; non-HER targeted TK inhibitors such as imatinib mesylate; multi-targeted tyrosine kinase inhibitors such as sunitinib; VEGF receptor tyrosine kinase inhibitors such as vatalanib (PTK787/ZK222584, available from Novartis/Schering AG); MAPK extracellular regulated kinase I inhibitor CI-1040 (available from Pharmacia); quinazolines, such as PD 153035,4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo[2,3-d] pyrimidines; curcumin (diferuloyl methane, 4,5-bis(4-fluoroanilino)phthalimide); tyrphostines containing nitrothiophene moieties; PD-0183805 (Warner-Lamber); antisense molecules (e.g. those that bind to HER-encoding nucleic acid); quinoxalines (U.S. Pat. No. 5,804,396); tryphostins (U.S. Pat. No. 5,804,396); ZD6474 (Astra Zeneca); PTK-787 (Novartis/Schering AG); pan-HER inhibitors such as CI-1033 (Pfizer); Affinitac (ISIS 3521; Isis/Lilly); imatinib mesylate (GLEEVEC®); PKI 166 (Novartis); GW2016 (Glaxo SmithKline); CI-1033 (Pfizer); EKB-569 (Wyeth); Semaxinib (Pfizer); ZD6474 (AstraZeneca); PTK-787 (Novartis/Schering AG); INC-1C11 (Imclone), rapamycin (sirolimus, RAPAMUNE®); or as described in any of the following patent publications: U.S. Pat. No. 5,804,396; WO 1999/09016 (American Cyanamid); WO 1998/43960 (American Cyanamid); WO 1997/38983 (Warner Lambert); WO 1999/06378 (Warner Lambert); WO 1999/06396 (Warner Lambert); WO 1996/30347 (Pfizer, Inc); WO 1996/33978 (Zeneca); WO 1996/3397 (Zeneca) and WO 1996/33980 (Zeneca). Each of the above-described references is incorporated herein by reference in its entirety for all of its teachings, including without limitation all methods, compounds, compositions, data, and the like, for use with any of the embodiments and disclosure herein.

Chemotherapeutic agents also include dexamethasone, interferons, colchicine, metoprine, cyclosporine, amphotericin, metronidazole, alemtuzumab, alitretinoin, allopurinol, amifostine, arsenic trioxide, asparaginase, BCG live, bevacuzimab, bexarotene, cladribine, clofarabine, darbepoetin alfa, denileukin, dexrazoxane, epoetin alfa, elotinib, filgrastim, histrelin acetate, ibritumomab, interferon alfa-2a, interferon alfa-2b, lenalidomide, levamisole, mesna, methoxsalen, nandrolone, nelarabine, nofetumomab, oprelvekin, palifermin, pamidronate, pegademase, pegaspargase, pegfilgrastim, pemetrexed disodium, plicamycin, porfimer sodium, quinacrine, rasburicase, sargramostim, temozolomide, VM-26, 6-TG, toremifene, tretinoin, ATRA, valrubicin, zoledronate, and zoledronic acid, and pharmaceutically acceptable salts thereof.

Chemotherapeutic agents also include hydrocortisone, hydrocortisone acetate, cortisone acetate, tixocortol pivalate, triamcinolone acetonide, triamcinolone alcohol, mometasone, amcinonide, budesonide, desonide, fluocinonide, fluocinolone acetonide, betamethasone, betamethasone sodium phosphate, dexamethasone, dexamethasone sodium phosphate, fluocortolone, hydrocortisone-17-butyrate, hydrocortisone-17-valerate, aclometasone dipropionate, betamethasone valerate, betamethasone dipropionate, prednicarbate, clobetasone-17-butyrate, clobetasol-17-propionate, fluocortolone caproate, fluocortolone pivalate and fluprednidene acetate; immune selective anti-inflammatory peptides (ImSAIDs) such as phenylalanine-glutamine-glycine (FEG) and its D-isomeric form (feG) (IMULAN Bioherapeutics, LLC); anti-rheumatic drugs such as azathioprine, ciclosporin (cyclosporine A), D-penicillamine, gold salts, hydroxychloroquine, leflunomideminocycline, sulfasalazine, tumor necrosis factor alpha (TNFα) blockers such as etanercept (Enbrel), infliximab (Remicade), adalimumab (Humira), certolizumab pegol (Cimzia), golimumab (Simponi), Interleukin 1 (IL-1) blockers such as anakinra (Kineret), T cell costimulation blockers such as abatacept (Orencia), Interleukin 6 (IL-6) blockers such as tocilizumab (ACTEMERA®); Interleukin 13 (IL-13) blockers such as lebrikizumab; Interferon alpha (IFN) blockers such as Rontalizumab; Beta 7 integrin blockers such as rhuMAb Beta7; IgE pathway blockers such as Anti-M1 prime; Secreted homotrimeric LTa3 and membrane bound heterotrimer LTal/02 blockers such as Anti-lymphotoxin alpha (LTa); radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); miscellaneous investigational agents such as thioplatin, PS-341, phenylbutyrate, ET-18-OCH₃, or famesyl transferase inhibitors (L-739749, L-744832); polyphenols such as quercetin, resveratrol, piceatannol, epigallocatechine gallate, theaflavins, flavanols, procyanidins, betulinic acid and derivatives thereof; autophagy inhibitors such as chloroquine; delta-9-tetrahydrocannabinol (dronabinol, MARINOL®); beta-lapachone; lapachol; colchicines; betulinic acid; acetylcamptothecin, scopolectin, and 9-aminocamptothecin); podophyllotoxin; tegafur; bexarotene; bisphosphonates such as clodronate, etidronate, NE-58095, zoledronic acid/zoledronate, alendronate, pamidronate, tiludronate, or risedronate; and epidermal growth factor receptor (EGF-R); vaccines such as THERATOPE® vaccine; perifosine, COX-2 inhibitor (e.g. celecoxib or etoricoxib), proteosome inhibitor (e.g. PS341); CCI-779; tipifamib (RI1577); orafenib, ABT510; Bcl-2 inhibitor such as oblimersen sodium; pixantrone; famesyltransferase inhibitors such as lonafamib (SCH 6636); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with oxaliplatin combined with 5-FU and leucovorin.

Chemotherapeutic agents also include non-steroidal anti-inflammatory drugs with analgesic, antipyretic, and anti-inflammatory effects. NSAIDs include non-selective inhibitors of the enzyme cyclooxygenase. Specific examples of NSAIDs include aspirin, propionic acid derivatives such as ibuprofen, fenoprofen, ketoprofen, flurbiprofen, oxaprozin and naproxen, acetic acid derivatives such as indomethacin, sulindac, etodolac, diclofenac, enolic acid derivatives such as piroxicam, meloxicam, tenoxicam, droxicam, lomoxicam and isoxicam, fenamic acid derivatives such as mefenamic acid, meclofenamic acid, flufenamic acid, tolfenamic acid, and COX-2 inhibitors such as celecoxib, etoricoxib, lumiracoxib, parecoxib, rofecoxib, rofecoxib, and valdecoxib. NSAIDs can be indicated for the symptomatic relief of conditions such as rheumatoid arthritis, osteoarthritis, inflammatory arthropathies, ankylosing spondylitis, psoriatic arthritis, Reiter's syndrome, acute gout, dysmenorrhoea, metastatic bone pain, headache and migraine, postoperative pain, mild-to-moderate pain due to inflammation and tissue injury, pyrexia, ileus, and renal colic.

In certain embodiments, chemotherapeutic agents include, but are not limited to, doxorubicin, dexamethasone, vincristine, cyclophosphamide, fluorouracil, topotecan, interferons, platinum derivatives, taxanes (e.g., paclitaxel, docetaxel), vinca alkaloids (e.g., vinblastine), anthracyclines (e.g., doxorubicin), epipodophyllotoxins (e.g., etoposide), cisplatin, an mTOR inhibitor (e.g., a rapamycin), methotrexate, actinomycin D, dolastatin 10, colchicine, trimetrexate, metoprine, cyclosporine, daunorubicin, teniposide, amphotericin, alkylating agents (e.g., chlorambucil), 5-fluorouracil, campthothecin, cisplatin, metronidazole, and imatinib mesylate, among others. In other embodiments, a compound disclosed herein is administered in combination with a biologic agent, such as bevacizumab or panitumumab.

In certain embodiments, compounds disclosed herein, or a pharmaceutically acceptable composition thereof, are administered in combination with an antiproliferative or chemotherapeutic agent selected from any one or more of abarelix, aldesleukin, alemtuzumab, alitretinoin, allopurinol, altretamine, amifostine, anastrozole, arsenic trioxide, asparaginase, azacitidine, BCG live, bevacuzimab, fluorouracil, bexarotene, bleomycin, bortezomib, busulfan, calusterone, capecitabine, camptothecin, carboplatin, carmustine, cetuximab, chlorambucil, cladribine, clofarabine, cyclophosphamide, cytarabine, dactinomycin, darbepoetin alfa, daunorubicin, denileukin, dexrazoxane, docetaxel, doxorubicin (neutral), doxorubicin hydrochloride, dromostanolone propionate, epirubicin, epoetin alfa, elotinib, estramustine, etoposide phosphate, etoposide, exemestane, filgrastim, floxuridine, fludarabine, fulvestrant, gefitinib, gemcitabine, gemtuzumab, goserelin acetate, histrelin acetate, hydroxyurea, ibritumomab, idarubicin, ifosfamide, imatinib mesylate, interferon alfa-2a, interferon alfa-2b, irinotecan, lenalidomide, letrozole, leucovorin, leuprolide acetate, levamisole, lomustine, megestrol acetate, melphalan, mercaptopurine, 6-MP, mesna, methotrexate, methoxsalen, mitomycin C, mitotane, mitoxantrone, nandrolone, nelarabine, nofetumomab, oprelvekin, oxaliplatin, paclitaxel, palifermin, pamidronate, pegademase, pegaspargase, pegfilgrastim, pemetrexed disodium, pentostatin, pipobroman, plicamycin, porfimer sodium, procarbazine, quinacrine, rasburicase, rituximab, sargramostim, sorafenib, streptozocin, sunitinib maleate, talc, tamoxifen, temozolomide, teniposide, VM-26, testolactone, thioguanine, 6-TG, thiotepa, topotecan, toremifene, tositumomab, trastuzumab, tretinoin, ATRA, uracil mustard, valrubicin, vinblastine, vincristine, vinorelbine, zoledronate, or zoledronic acid.

### Cancers

Disclosed herein are methods of treating cancer using a combination disclosed herein.

“Cancer” refers to all types of cancer, neoplasm or malignant tumors found in mammals (e.g. humans), including, without limitation, leukemias, lymphomas, myelomas, carcinomas, and sarcomas. Exemplary cancers that may be treated with a compound or method provided herein include brain cancer, glioma, glioblastoma, neuroblastoma, prostate cancer, colorectal cancer, pancreatic cancer (such as pancreatic adenocarcinoma, PDAC), medulloblastoma, melanoma, cervical cancer, gastric cancer, ovarian cancer, lung cancer, cancer of the head, Hodgkin's Disease, and Non-Hodgkin's Lymphomas. Exemplary cancers that may be treated with a compound or method provided herein include cancer of the blood, thyroid, endocrine system, brain, breast, cervix, colon, head & neck, liver, kidney, lung, ovary, pancreas, rectum, stomach, and uterus. Additional examples include, thyroid carcinoma, cholangiocarcinoma, pancreatic adenocarcinoma, skin cutaneous melanoma, colon adenocarcinoma, rectum adenocarcinoma, stomach adenocarcinoma, esophageal carcinoma, head and neck squamous cell carcinoma, breast invasive carcinoma, lung adenocarcinoma, lung squamous cell carcinoma, non-small cell lung carcinoma, mesothelioma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

The methods comprising the combination therapies disclosed herein can be use in connection with any cancer having a FLT3 mutation, for example mutations that cause constitutive activation.

In some embodiments, the cancer harbors at least one of a EGFR, KRAS, BRAF (e.g., BRAF class III) and/or NF1 (e.g., loss of function) mutations.

In some embodiments, the methods disclosed herein are suitable for the treatment of any cancer in which there is a mutation in FLT3. In embodiments, the cancer is a hematologic malignancy. In embodiments, the cancer is acute myeloid leukemia. In embodiments, the cancer is colorectal cancer. In embodiments, the cancer is colon cancer (e.g., colon adenocarcinoma). In embodiments, the cancer is lung cancer (e.g., lung adenocarcinoma). In embodiments, the cancer is melanoma (e.g., cutaneous melanoma). In embodiments, the cancer is breast cancer (e.g., breast invasive ductal carcinoma). As will be appreciated by those skilled in the art, tumors may metastasize from a first or primary locus of tumor to one or more other body tissues or sites. In particular, metastases to the central nervous system (i.e., secondary CNS tumors), and particularly the brain (i.e., brain metastases), are well documented for tumors and cancers, such as breast, lung, melanoma, renal and colorectal. As such, the methods disclosed herein can be used for the treatment of metastases (i.e., metastatic tumor growth) to other organs as well.

In some embodiments, the cancer has a class 1 B-Raf mutation.

In some embodiments, the mutant B-Raf comprises a V600 mutation. In some embodiments, the mutant of B-Raf comprises the mutation V600E. In some embodiments, the mutation is V600K. In some embodiments, the mutation is V600D. In some embodiments, the mutation is V600L. In some embodiments, the mutation is V600R. In some embodiments, the cancer is a BRAF V600E or V600K mutant tumor.

In some embodiments, the cancer is a mitogen-activated protein kinase (MAPK) pathway driven cancer.

In some embodiments, the cancer is a BRAF-driven cancer, HRAS-driven cancer, or a NRAS-driven cancer.

In some embodiments, the cancer comprises at least one cancer cell driven by deregulated ERK.

In some embodiments, the cancer has at least one mutation in RAS. In some embodiments, the cancer has at least one mutation in RAF. In some embodiments, the cancer has at least one mutation in MEK.

In some embodiments, the cancer has a G12C KRAS mutation. In some embodiments, the cancer has a G12D KRAS mutation. In some embodiments, the cancer has a G12S KRAS mutation. In some embodiments, the cancer has a G12V KRAS mutation. In some embodiments, the cancer has a G13D KRAS mutation. In some embodiments, the cancer has a Q16H KRAS mutation. In some embodiments, the cancer has a Q16K KRAS mutation. In some embodiments, the cancer has a Q61R NRAS mutation.

In some embodiments, the cancer is a MAPKm/MAPKi-naïve pan cancer.

In some embodiments, the cancer comprises one or more EGFR mutation selected from the group consisting of EGFR gene copy gain, EGFR gene amplification, chromosome 7 polysomy, L858R, exon 19 deletions/insertions, L861Q, G719C, G719S, G719A, V765A, T783A, exon 20 insertions, EGFR splice variants (Viii, Vvi, and Vii), A289D, A289T, A289V, G598A, G598V, T790M, and C797S. In some embodiments, the cancer comprises one or more EGFR mutation selected from the group consisting of L858R, exon 19 deletion, and T790M.

In some embodiments, the cancer is a liquid tumor, hematologic malignancy, or a blood cancer.

In some embodiments, the cancer is a leukemia, lymphoma, or melanoma. In some embodiments, the cancer is acute myeloid leukemia (AML). In some embodiments, the cancer is the AML is relapsed and/or refractory AML. In some embodiments, the cancer is the AML is a FLT3 mutant AML.

In some embodiments, the cancer is a solid tumor. In some embodiments, the solid tumor is an advanced or a metastatic solid tumor.

In some embodiments, the cancer is non-small cell lung cancer (NSCLC), melanoma, pancreatic cancer (such as pancreatic cancer is pancreatic ductal adenocarcinoma, PDAC), salivary gland tumor, thyroid cancer, colorectal cancer (CRC), or esophageal cancer.

In some embodiments, the cancer is non-small cell lung cancer (NSCLC). In some embodiments, the NSCLC is an EGFR mutant NSCLC. In some embodiments, the NSCLC is a KRAS G12C mutant NSCLC. In some embodiments, the NSCLC is a KRAS G12D mutant NSCLC. In some embodiments, the NSCLC is a KRAS G12S mutant NSCLC. In some embodiments, the NSCLC is a KRAS G12V mutant NSCLC. In some embodiments, the NSCLC is a KRAS G13D mutant NSCLC. In some embodiments, the NSCLC is a KRAS Q61H mutant NSCLC. In some embodiments, the NSCLC is a KRAS Q61K mutant NSCLC.

In some embodiments, the NSCLC is a NRAS Q61R mutant NSCLC. In some embodiments, the cancer is a MAPKm/MAPKi-naïve NSCLC. In some embodiments, the cancer is a BRAFi-treated V600 NSCLC. In some embodiments, the cancer is a KRAS-treated G12C NSCLC. In some embodiments, the cancer is a KRAS-treated G12D NSCLC. In some embodiments, the cancer is a KRAS-treated G12S NSCLC. In some embodiments, the cancer is a KRAS-treated G12V NSCLC. In some embodiments, the cancer is a KRAS-treated G13D NSCLC. In some embodiments, the cancer is a KRAS-treated Q61H NSCLC. In some embodiments, the cancer is a KRAS-treated Q61K NSCLC. In some embodiments, the cancer is a NRAS-treated Q61R NSCLC.

In some embodiments, the cancer is pancreatic cancer. In some embodiments, the cancer is a MAPKm/MAPKi-naïve pancreatic cancer. In some embodiments, the pancreatic cancer is pancreatic ductal adenocarcinoma (PDAC). In some embodiments, the cancer is a G12V mutant PDAC.

In some embodiments, the cancer is melanoma. In some embodiments, the melanoma is a BRAF V600E or V600K mutant tumor. In some embodiments, the cancer is a BRAFi-treated V600 melanoma.

In some embodiments, the cancer is salivary gland tumor.

In some embodiments, the cancer is thyroid cancer.

In some embodiments, the cancer is colorectal cancer (CRC). In some embodiments, the CRC is a BRAF V600E CRC. In some embodiments, the CRC is a KRAS mutant CRC.

In some embodiments, the CRC is a KRAS G12C mutant CRC. In some embodiments, the CRC is a KRAS G12D mutant CRC. In some embodiments, the CRC is a KRAS G12S mutant CRC. In some embodiments, the CRC is a KRAS G12V mutant CRC. In some embodiments, the CRC is a KRAS G13D mutant CRC. In some embodiments, the CRC is a KRAS Q61H mutant CRC. In some embodiments, the CRC is a KRAS Q61K mutant CRC. In some embodiments, the CRC is a NRAS mutant CRC. In some embodiments, the CRC is a NRAS Q61R mutant CRC.

In some embodiments, the cancer is esophageal cancer.

### Dosing

In one aspect, the compositions described herein are used for the treatment of diseases and conditions described herein. In addition, a method for treating any of the diseases or conditions described herein in a subject in need of such treatment, involves administration of compositions in therapeutically effective amounts to said subject.

Dosages of compositions described herein can be determined by any suitable method. Maximum tolerated doses (MTD) and maximum response doses (MRD) for compound 1, or a pharmaceutically acceptable salt thereof can be determined via established animal and human experimental protocols as well as in the examples described herein. For example, toxicity and therapeutic efficacy of compound 1, or a pharmaceutically acceptable salt thereof, can be determined by standard pharmaceutical procedures in cell cultures or experimental animals, including, but not limited to, for determining the LDso (the dose lethal to 50% of the population) and the ED₅₀ (the dose therapeutically effective in 50% of the population). The dose ratio between the toxic and therapeutic effects is the therapeutic index and it can be expressed as the ratio between LDso and ED₅₀. The data obtained from cell culture assays and animal studies can be used in formulating a range of dosage for use in human. The dosage of such compounds lies preferably within a range of circulating concentrations that include the ED₅₀ with minimal toxicity.

The dosage may vary within this range depending upon the dosage form employed and the route of administration utilized. Additional relative dosages, represented as a percent of maximal response or of maximum tolerated dose, are readily obtained via the protocols.

In some embodiments, the amount of a given formulation comprising compound 1, or a pharmaceutically acceptable salt thereof that corresponds to such an amount varies depending upon factors such as the molecular weight of a particular salt or form, disease condition and its severity, the identity (e.g., age, weight, sex) of the subject or host in need of treatment, but can nevertheless be determined according to the particular circumstances surrounding the case, including, e.g., the specific agent being administered, the liquid formulation type, the condition being treated, and the subject or host being treated.

In some embodiments, gilteritinib is administered in an amount that is between about 100 mg/day and 500 mg/day. In some embodiments, gilteritinib is administered in an amount that is about 120 mg/day.

In some embodiments, the amount of compound 1, or a pharmaceutically acceptable salt thereof, as described herein is relative to the free-base equivalent of compound 1.

In some embodiments, compound 1, or a pharmaceutically acceptable salt thereof, is administered orally.

In some embodiments, compound 1, or a pharmaceutically acceptable salt thereof, is administered in an amount that is between about 25 mg/day and about 300 mg/day.

In some embodiments, compound 1, or a pharmaceutically acceptable salt thereof, is administered in an amount that is between 25 mg/day and 150 mg/day.

In some embodiments, compound 1, or a pharmaceutically acceptable salt thereof, is administered in an amount that is about 25 mg/day, about 50 mg/day, about 75 mg/day, about 100 mg/day, about 125 mg/day, about 150 mg/day, about 175 mg/day, about 200 mg/day, about 225 mg/day, or about 250 mg/day.

In some embodiments, compound 1, or a pharmaceutically acceptable salt thereof, is administered in an amount that is about 25 mg/day, about 50 mg/day, about 100 mg/day, or about 150 mg/day.

In some embodiments, compound 1, or a pharmaceutically acceptable salt thereof, is administered in an amount between about 25 mg to about 300 mg twice a day, once a week (BID-QW).

In some embodiments, compound 1, or a pharmaceutically acceptable salt thereof, is administered in an amount that is between about 25 mg and about 250 mg twice a day, once a week (BID-QW).

In some embodiments, compound 1, or a pharmaceutically acceptable salt thereof, is administered in an amount that is between about 25 mg and about 200 twice a day, once a week (BID-QW).

In some embodiments, compound 1, or a pharmaceutically acceptable salt thereof, is administered in an amount that is between about 25 mg and about 150 mg twice a day, once a week (BID-QW).

In some embodiments, compound 1, or a pharmaceutically acceptable salt thereof, is administered in an amount that is between about 25 mg and about 100 mg twice a day, once a week (BID-QW).

In some embodiments, compound 1, or a pharmaceutically acceptable salt thereof, is administered in an amount that is between about 25 mg and about 50 mg twice a day, once a week (BID-QW).

In some embodiments, compound 1, or a pharmaceutically acceptable salt thereof, is administered in an amount that is between about 50 mg to about 300 mg twice a day, once a week (BID-QW).

In some embodiments, compound 1, or a pharmaceutically acceptable salt thereof, is administered in an amount that is between about 50 mg and about 250 twice a day, once a week (BID-QW).

In some embodiments, compound 1, or a pharmaceutically acceptable salt thereof, is administered in an amount that is between about 50 mg and about 200 mg twice a day, once a week (BID-QW).

In some embodiments, compound 1, or a pharmaceutically acceptable salt thereof, is administered in an amount that is between about 50 mg and about 150 mg twice a day, once a week (BID-QW).

In some embodiments, compound 1, or a pharmaceutically acceptable salt thereof, is administered in an amount that is between about 50 mg and about 125 mg twice a day, once a week (BID-QW).

In some embodiments, compound 1, or a pharmaceutically acceptable salt thereof, is administered in an amount that is between about 50 mg and about 100 mg twice a day, once a week (BID-QW).

In some embodiments, compound 1, or a pharmaceutically acceptable salt thereof, is administered in an amount that is between about 100 mg and about 300 mg twice a day, once a week (BID-QW).

In some embodiments, compound 1, or a pharmaceutically acceptable salt thereof, is administered in an amount that is between about 100 mg and about 250 mg twice a day, once a week (BID-QW).

In some embodiments, compound 1, or a pharmaceutically acceptable salt thereof, is administered in an amount that is between about 100 mg and about 200 mg twice a day, once a week (BID-QW).

In some embodiments, compound 1, or a pharmaceutically acceptable salt thereof, is administered in an amount that is between about 100 mg and about 150 mg twice a day, once a week (BID-QW).

In some embodiments, compound 1, or a pharmaceutically acceptable salt thereof, is administered in an amount that is between about 150 mg and about 300 mg twice a day, once a week (BID-QW).

In some embodiments, compound 1, or a pharmaceutically acceptable salt thereof, is administered in an amount that is between about 150 mg and about 250 mg twice a day, once a week (BID-QW).

In some embodiments, compound 1, or a pharmaceutically acceptable salt thereof, is administered in an amount that is between about 150 mg and about 200 mg twice a day, once a week (BID-QW).

In some embodiments, compound 1, or a pharmaceutically acceptable salt thereof, is administered in an amount that is between about 175 mg and about 300 mg twice a day, once a week (BID-QW).

In some embodiments, compound 1, or a pharmaceutically acceptable salt thereof, is administered in an amount that is between about 175 mg and about 250 mg twice a day, once a week (BID-QW).

In some embodiments, compound 1, or a pharmaceutically acceptable salt thereof, is administered in an amount that is between about 175 mg and about 200 mg twice a day, once a week (BID-QW).

In some embodiments, compound 1, or a pharmaceutically acceptable salt thereof, is administered in an amount that is between about 200 mg and about 300 mg twice a day, once a week (BID-QW).

In some embodiments, compound 1, or a pharmaceutically acceptable salt thereof, is administered in an amount that is between about 200 mg and about 250 mg twice a day, once a week (BID-QW).

In some embodiments, compound 1, or a pharmaceutically acceptable salt thereof, is administered in an amount that is between about 225 mg and about 300 mg twice a day, once a week (BID-QW).

In some embodiments, compound 1, or a pharmaceutically acceptable salt thereof, is administered in an amount that is between about 225 mg and about 250 mg twice a day, once a week (BID-QW).

In some embodiments, compound 1, or a pharmaceutically acceptable salt thereof, is administered in an amount that is between about 25 mg and about 300 mg twice a day, once a week (BID-QW).

In some embodiments, compound 1, or a pharmaceutically acceptable salt thereof, is administered in an amount that is between about 25 mg and about 250 mg twice a day, once a week (BID-QW).

In some embodiments, compound 1, or a pharmaceutically acceptable salt thereof, is administered in an amount that is between about 25 mg and about 150 mg twice a day, once a week (BID-QW).

In some embodiments, compound 1, or a pharmaceutically acceptable salt thereof, is administered in an amount that is about 25 mg, 50 mg, about 75 mg, about 100 mg, about 125 mg, about 150 mg, about 175 mg, about 200 mg, about 225 mg, or about 250 mg twice a day, once a week (BID-QW).

In some embodiments, compound 1, or a pharmaceutically acceptable salt thereof, is administered in an amount that is about 25 mg, 50 mg, about 100 mg, about 125 mg, or about 150 mg twice a day, once a week (BID-QW).

In some embodiments, compound 1, or a pharmaceutically acceptable salt thereof, is administered in an amount that is about 125 mg twice a day, once a week (BID-QW).

In some embodiments, compound 1, or a pharmaceutically acceptable salt thereof, is administered in an amount that is about 250 mg once a day, once a week.

In some embodiments, compound 1, or a pharmaceutically acceptable salt thereof, is administered in an amount that is between about 25 mg and about 300 mg once a week (QW).

In some embodiments, compound 1, or a pharmaceutically acceptable salt thereof, is administered in an amount that is between about 50 mg and about 250 mg once a week (QW).

In some embodiments, compound 1, or a pharmaceutically acceptable salt thereof, is administered in an amount that is between about 100 mg and about 300 mg once a week (QW).

In some embodiments, compound 1, or a pharmaceutically acceptable salt thereof, is administered in an amount that is between about 100 mg and about 250 mg once a week (QW).

In some embodiments, compound 1, or a pharmaceutically acceptable salt thereof, is administered in an amount that is between about 125 mg and about 250 mg once a week (QW).

In some embodiments, compound 1, or a pharmaceutically acceptable salt thereof, is administered in an amount that is between about 150 mg and about 300 mg once a week (QW).

In some embodiments, compound 1, or a pharmaceutically acceptable salt thereof, is administered in an amount that is between about 150 mg and about 250 mg once a week (QW).

In some embodiments, compound 1, or a pharmaceutically acceptable salt thereof, is administered in an amount that is about 100 mg once a week (QW). In some embodiments, compound 1, or a pharmaceutically acceptable salt thereof, is administered in an amount that is about 150 mg once a week (QW). In some embodiments, compound 1, or a pharmaceutically acceptable salt thereof, is administered in an amount that is about 200 mg once a week (QW). In some embodiments, compound 1, or a pharmaceutically acceptable salt thereof, is administered in an amount that is about 250 mg once a week (QW).

In some embodiments, compound 1, or a pharmaceutically acceptable salt thereof, is administered in an amount that is about 25 mg, 30 mg, 40 mg, 50 mg, about 60 mg, about 65 mg about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 140 mg, about 150 mg, about 160 mg, about 170 mg, about 175 mg, about 180 mg, about 190 mg, about 200 mg, about 210 mg, about 220 mg, about 225 mg, about 230 mg, about 240 mg, about 250 mg, about 260 mg, about 270 mg, about 280 mg, about 290 mg, or about 300 mg.

In some embodiments, each of the above-recited amounts may be administered QD, QW, BID, BID-QD, or BID-QW.

In some embodiments, the dosing of compound 2 can be in any suitable amount to treat the cancer. For example, the dosing could be a daily dosage of between 1 mg up to 500 mg. As an additional example, the daily dose could be in a range from about 20 mg to 400 mg (or any sub-range or sub-value there between, including endpoints). In some embodiments, the range of dosing of compound 2 can be from 10 mg to 300 mg. In some embodiments, the range of dosing of compound 2 can be from 10 mg to 100 mg. In some embodiments, the range of dosing of compound 2 can be from 5 mg to 50 mg. In some embodiments, compound 2 is administered in an amount that is about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, or about 150 mg. The daily dosage can be achieved by administering a single administered dosage (e.g., QD) or via multiple administrations during a day (e.g., BID, TID, QID, etc.) to provide the total daily dosage. In some embodiments, compound 2 is administered once a day QD. In some embodiments, compound 2 is administered once daily (QD) for 21 days followed by a 7-day break (3 weeks on, 1 week off) on a 28-day cycle. In some embodiments, compound 2 is administered twice a day (BID). In some embodiments, compound 2 is administered twice a day for 21 days followed by a 7-day break (3 weeks on, 1 week off) on a 28-day cycle. In embodiments, the dosing of the FLT3 inhibitor is any suitable amount. For example, it can be an amount in a range from 1 mg to 500 mg daily (or any sub-range or sub-value there between, including endpoints).

In some embodiments, compound 2 is administered QD or BID for 2 weeks on and 1 week off (21 day schedule). In some embodiments, compound 2 is administered QD or BID for 3 weeks on and 1 week off (28 day schedule). In some embodiments, compound 2 is administered QD or BID three times a week (D1D3D5 TIW) e.g., Day 1, Day 3, and Day 5. In some embodiments, compound 2 is administered twice a day/twice a week e.g., Day 1 and Day 2 (BID-D1D2-BIW).

In some embodiments, compound 2 is administered once a day (QD) continuous dosing at a dose of 20 mg/day to 60 mg/day, 40 mg/day, or 60 mg/day. In some embodiments, compound 2 is administered twice a day (BID) continuous dosing at a dose of 20 mg/day to 80 mg/day. In some embodiments, compound 2 is administered twice a day (BID) continuous dosing at a dose of 10 mg/day to 100 mg/day.

Dosing of the FLT3 inhibitor may be the same or less than the approved dosing for any given FLT3 inhibitor and may depend on a given indication. In some embodiments, gilteritinib may be administered from 100 mg to 800 mg daily. For example, gilteritinib has been approved at a dose of 120 mg daily. In some embodiments, the FLT3 inhibitor is administered in an amount that is about 120 mg. In some embodiments, the FLT3 inhibitor is administered once a day (QD). Gilteritinib has also been approved reduced dose such as 80 mg daily. It will be appreciated that each of the recited ranges above can include any sub-range or sub-point therein, inclusive of endpoints. It will be appreciated that each of the recited ranges above can include any sub-range or sub-point therein, inclusive of endpoints. A common dose range for adult humans is generally from 5 mg to 2 g/day. Tablets or other forms of presentation provided in discrete units may conveniently contain an amount of one or more compounds which is effective at such dosage or as a multiple of the same, for instance, units containing 5 mg to 500 mg, usually around 10 mg to 200 mg.

### Administration

Administration of compound 1 or compound 2, or a pharmaceutically acceptable salt thereof, and combination partners described herein are at a dosage described herein or at other dose levels and compositions determined and contemplated by a medical practitioner. In certain embodiments, compound 1 or compound 2, or a pharmaceutically acceptable salt thereof, is administered for prophylactic and/or therapeutic treatments. In certain therapeutic applications, compound 1 or compound 2, or a pharmaceutically acceptable salt thereof, and combination partners described herein, are administered to a patient already suffering from a disease in an amount sufficient to cure the disease or at least partially arrest or ameliorate the symptoms. Amounts effective for this use depend on the age of the patient, severity of the disease, previous therapy, the patient's health status, weight, and response to the compositions, and the judgment of the treating physician. Therapeutically effective amounts are optionally determined by methods including, but not limited to, a dose escalation clinical trial.

In prophylactic applications, the compositions described herein are administered to a patient susceptible to or otherwise at risk of a particular disease, e.g., cancer. Such an amount is defined to be a “prophylactically effective amount or dose.” In this use, the precise amounts also depend on the patient's age, state of health, weight, and the like. When used in a patient, effective amounts for this use will depend on the risk or susceptibility of developing the particular disease, previous therapy, the patient's health status and response to the compositions, and the judgment of the treating physician.

In certain embodiments wherein the patient's condition does not improve, upon the doctor's discretion the administration of a composition described herein are administered chronically, that is, for an extended period of time, including throughout the duration of the patient's life in order to ameliorate or otherwise control or limit the symptoms of the patient's disease. In other embodiments, administration of a composition continues until complete or partial response of a disease.

In some embodiments, compound 1 or compound 2, or a pharmaceutically acceptable salt thereof, and combination partners described herein, are administered once a day. In some embodiments, compound 1 or compound 2, or a pharmaceutically acceptable salt thereof, and combination partners described herein are administered twice a day. In some embodiments, compound 1 or compound 2, or a pharmaceutically acceptable salt thereof, and combination partners described herein are administered three times a day.

In some embodiments, gilteritinib is administered once daily. In some embodiments, gilteritinib is administered twice daily. In some embodiments, gilteritinib is administered three times daily.

In some embodiments, compound 1 or compound 2, or a pharmaceutically acceptable salt thereof, and combination partners described herein are administered to a subject who is in a fasted state. A fasted state refers to a subject who has gone without food or fasted for a certain period of time. General fasting periods include at least 4 hours, at least 6 hours, at least 8 hours, at least 10 hours, at least 12 hours, at least 14 hours and at least 16 hours without food. In some embodiments, compound 1 or compound 2, or a pharmaceutically acceptable salt thereof, is administered to a subject who is in a fasted state for at least 8 hours. In other embodiments, compound 1 or compound 2, or a pharmaceutically acceptable salt thereof, and combination partners described herein, are administered to a subject who is in a fasted state for at least 10 hours. In yet other embodiments, compound 1 or compound 2, or a pharmaceutically acceptable salt thereof, and combination partners described herein, are administered to a subject who is in a fasted state for at least 12 hours. In other embodiments, compound 1 or compound 2, or a pharmaceutically acceptable salt thereof, and combination partners described herein, are administered to a subject who has fasted overnight.

In other embodiments, compound 1 or compound 2, or a pharmaceutically acceptable salt thereof, and combination partners described herein, are administered to a subject who is in a fed state. A fed state refers to a subject who has taken food or has had a meal. In certain embodiments, a composition is administered to a subject in a fed state 5 minutes post-meal, 10 minutes post-meal, 15 minutes post-meal, 20 minutes post-meal, 30 minutes post-meal, 40 minutes post-meal, 50 minutes post-meal, 1 hour post-meal, or 2 hours post-meal. In certain instances, compound 1 or compound 2, or a pharmaceutically acceptable salt thereof, is administered to a subject in a fed state 30 minutes post-meal. In other instances, compound 1 or compound 2, or a pharmaceutically acceptable salt thereof, and combination partners described herein, are administered to a subject in a fed state 1 hour post-meal. In yet further embodiments, compound 1 or compound 2, or a pharmaceutically acceptable salt thereof, is administered to a subject with food.

The length of a treatment cycle depends on the treatment being given. In some embodiments, the length of a treatment cycle ranges from two to six weeks. In some embodiments, the length of a treatment cycle ranges from three to six weeks. In some embodiments, the length of a treatment cycle ranges from three to four weeks. In some embodiments, the length of a treatment cycle is three weeks (or 21 days). In some embodiments, the length of a treatment cycle is four weeks (28 days). In some embodiments, the length of a treatment cycle is five weeks (35 days). In some embodiments, the length of a treatment cycle is 56 days. In some embodiments, a treatment cycle lasts one, two, three, four, or five weeks. In some embodiments, a treatment cycle lasts three weeks. In some embodiments, a treatment cycle lasts four weeks. In some embodiments, a treatment cycle lasts five weeks. The number of treatment doses scheduled within each cycle also varies depending on the drugs being given.

In some embodiments of a method of treating cancer, compound 1 or compound 2, or a pharmaceutically acceptable salt thereof, and combination partners described herein are administered in 28-day cycles. In some embodiments of a method of treating cancer, compound 1 or compound 2, or a pharmaceutically acceptable salt thereof, and combination partners described herein, are administered for multiple 28-day cycles. In some embodiments of a method of treating cancer, compound 1 or compound 2, or a pharmaceutically acceptable salt thereof, and combination partners described herein, are administered for at least one 28-day cycle. In some embodiments of a method of treating cancer, compound 1 or compound 2, or a pharmaceutically acceptable salt thereof, and combination partners described herein, are administered for at least two 28-day cycles. In some embodiments of a method of treating cancer, compound 1 or compound 2, or a pharmaceutically acceptable salt thereof, and combination partners described herein, are administered for at least three 28-day cycles. In some embodiments of a method of treating cancer, compound 1 or compound 2, or a pharmaceutically acceptable salt thereof, and combination partners described herein, are administered for at least four 28-day cycles. In some embodiments of a method of treating cancer, compound 1 or compound 2, or a pharmaceutically acceptable salt thereof, and combination partners described herein, are administered for at least five 28-day cycles. In some embodiments of a method of treating cancer, compound 1 or compound 2, or a pharmaceutically acceptable salt thereof, and combination partners described herein, are administered for at least six 28-day cycles.

In some embodiments of a method of treating cancer, compound 1 or compound 2, or a pharmaceutically acceptable salt thereof, is administered on days 1-7 of each 28-day cycle. In some embodiments of a method of treating cancer, compound 1 or compound 2, or a pharmaceutically acceptable salt thereof, is administered on days 1-14 of each 28-day cycle. In some embodiments of a method of treating cancer, compound 1 or compound 2, or a pharmaceutically acceptable salt thereof, is administered on days 1-21 of each 28-day cycle. In some embodiments of a method of treating cancer, compound 1 or compound 2, or a pharmaceutically acceptable salt thereof, is administered on days 1-28 of each 28-day cycle.

In some embodiments of a method of treating cancer, compound 1 or compound 2, or a pharmaceutically acceptable salt thereof, is administered twice a day on day 1 of a 28-day cycle. In some embodiments of a method of treating cancer, compound 1 or compound 2, or a pharmaceutically acceptable salt thereof, is administered twice a day on day 8 of a 28-day cycle. In some embodiments of a method of treating cancer, compound 1 or compound 2, or a pharmaceutically acceptable salt thereof, is administered twice a day on day 15 of a 28-day cycle. In some embodiments of a method of treating cancer, compound 1 or compound 2, or a pharmaceutically acceptable salt thereof, is administered twice a day on day 22 of a 28-day cycle. In some embodiments of a method of treating cancer, compound 1 or compound 2, or a pharmaceutically acceptable salt thereof, is not administered twice a day on day 22 of a 28-day cycle.

In some embodiments of a method of treating cancer, compound 1 or compound 2, or a pharmaceutically acceptable salt thereof, is administered twice a day on day 1, day 8, and day 15 of a 28-day cycle.

In some embodiments of a method of treating cancer, compound 1 or compound 2, or a pharmaceutically acceptable salt thereof, is not administered on days 2-7, days 9-14, days 16-21, days 23-28 of a 28-day cycle.

In some embodiments of a method of treating cancer, compound 1 or compound 2, or a pharmaceutically acceptable salt thereof, and combination partners described herein are administered in 35-day cycles. In some embodiments of a method of treating cancer, compound 1 or compound 2, or a pharmaceutically acceptable salt thereof, and combination partners described herein are administered for multiple 35-day cycles. In some embodiments of a method of treating cancer, compound 1 or compound 2, or a pharmaceutically acceptable salt thereof, and combination partners described herein are administered for at least one 35-day cycle. In some embodiments of a method of treating cancer, compound 1 or compound 2, or a pharmaceutically acceptable salt thereof, and combination partners described herein are administered for at least two 35-day cycle. In some embodiments of a method of treating cancer, compound 1 or compound 2, or a pharmaceutically acceptable salt thereof, and combination partners described herein are administered for at least three 35-day cycle. In some embodiments of a method of treating cancer, compound 1 or compound 2, or a pharmaceutically acceptable salt thereof, and combination partners described herein are administered for at least four 35-day cycle. In some embodiments of a method of treating cancer, compound 1 or compound 2, or a pharmaceutically acceptable salt thereof, and combination partners described herein are administered for at least five 35-day cycle. In some embodiments of a method of treating cancer, compound 1 or compound 2, or a pharmaceutically acceptable salt thereof, and combination partners described herein are administered for at least six 35-day cycle.

In some embodiments of a method of treating cancer, compound 1 or compound 2, or a pharmaceutically acceptable salt thereof, is administered on days 1-7 of each 35-day cycle. In some embodiments of a method of treating cancer, compound 1 or compound 2, or a pharmaceutically acceptable salt thereof, is administered on days 1-14 of each 35-day cycle. In some embodiments of a method of treating cancer, compound 1 or compound 2, or a pharmaceutically acceptable salt thereof, is administered on days 1-21 of each 35-day cycle. In some embodiments of a method of treating cancer, compound 1 or compound 2, or a pharmaceutically acceptable salt thereof, is administered on days 1-28 of each 35-day cycle. In some embodiments of a method of treating cancer, compound 1 or compound 2, or a pharmaceutically acceptable salt thereof, is administered on days 1-35 of each 35-day cycle.

In some embodiments of a method of treating cancer, compound 1 or compound 2, or a pharmaceutically acceptable salt thereof, is administered twice a day on day 1 of a 35-day cycle. In some embodiments of a method of treating cancer, compound 1 or compound 2, or a pharmaceutically acceptable salt thereof, is administered twice a day on day 8 of a 35-day cycle. In some embodiments of a method of treating cancer, compound 1 or compound 2, or a pharmaceutically acceptable salt thereof, is administered twice a day on day 15 of a 35-day cycle. In some embodiments of a method of treating cancer, compound 1 or compound 2, or a pharmaceutically acceptable salt thereof, is administered twice a day on day 22 of a 35-day cycle. In some embodiments of a method of treating cancer, compound 1 or compound 2, or a pharmaceutically acceptable salt thereof, is administered twice a day on day 29 of a 35-day cycle. In some embodiments of a method of treating cancer, compound 1 or compound 2, or a pharmaceutically acceptable salt thereof, is not administered twice a day on day 29 of a 35-day cycle.

In some embodiments of a method of treating a cancer, compound 1 or compound 2, or a pharmaceutically acceptable salt thereof, is administered twice a day on day 1, day 8, day 15, and day 22 of a 35-day cycle.

In some embodiments of a method of treating cancer, compound 1 or compound 2, or a pharmaceutically acceptable salt thereof, is not administered on days 2-7, days 9-14, days 16-21, days 23-28, and days 30-35 of a 28-day cycle.

### Compositions

In some embodiments, there are provided pharmaceutical compositions comprising the compound disclosed herein and a pharmaceutically acceptable excipient. In some embodiments, the pharmaceutical compositions are configured as an oral tablet preparation.

The compounds disclosed herein can be prepared and administered in a wide variety of oral, parenteral, and topical dosage forms. Oral preparations include tablets, pills, powder, dragees, capsules, liquids, lozenges, gels, syrups, slurries, suspensions, etc., suitable for ingestion by the patient. The compounds disclosed herein can also be administered by injection, that is, intravenously, intramuscularly, intracutaneously, subcutaneously, intraduodenally, or intraperitoneally. Also, the compounds described herein can be administered by inhalation, for example, intranasally. Additionally, the compounds of the present embodiments can be administered transdermally. The compound disclosed herein can also be administered by in intraocular, intravaginal, and intrarectal routes including suppositories, insufflation, powders, and aerosol formulations (for examples of steroid inhalants, see Rohatagi, J. Clin. Pharmacol. 35:1187-1193, 1995; Tjwa, Ann. Allergy Asthma Immunol. 75:107-111, 1995), which is incorporated herein by reference in its entirety for all of its teachings, including without limitation all methods, compounds, compositions, data, and the like, for use with any of the embodiments and disclosure herein.

For preparing pharmaceutical compositions from the compounds disclosed herein, pharmaceutically acceptable carriers can be either solid or liquid. Solid form preparations include powders, tablets, pills, capsules, cachets, suppositories, and dispersible granules. A solid carrier can be one or more substances, which may also act as diluents, flavoring agents, surfactants, binders, preservatives, tablet disintegrating agents, or an encapsulating material. Details on techniques for formulation and administration are well described in the scientific and patent literature, see, e.g., the latest edition of Remington's Pharmaceutical Sciences, Maack Publishing Co, Easton PA (“Remington's”), which is incorporated herein by reference in its entirety for all of its teachings, including without limitation all methods, compounds, compositions, data, and the like, for use with any of the embodiments and disclosure herein.

In powders, the carrier is a finely divided solid, which is in a mixture with the finely divided active component. In tablets, the active component is mixed with the carrier having the necessary binding properties and additional excipients as required in suitable proportions and compacted in the shape and size desired.

The powders, capsules and tablets preferably contain from 5% or 10% to 70% of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other excipients, is surrounded by a carrier, which is thus in association with it. Similarly, cachets and lozenges are included. Tablets, powders, capsules, pills, cachets, and lozenges can be used as solid dosage forms suitable for oral administration.

Suitable solid excipients are carbohydrate or protein fillers including, but not limited to sugars, including lactose, sucrose, mannitol, or sorbitol; starch from corn, wheat, rice, potato, or other plants; cellulose such as methyl cellulose, hydroxypropylmethyl-cellulose, or sodium carboxymethylcellulose; and gums including arabic and tragacanth; as well as proteins such as gelatin and collagen. If desired, disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage). Pharmaceutical preparations disclosed herein can also be used orally using, for example, push-fit capsules made of gelatin, as well as soft, sealed capsules made of gelatin and a coating such as glycerol or sorbitol. Push-fit capsules can contain the compound disclosed herein mixed with a filler or binders such as lactose or starches, lubricants such as talc or magnesium stearate, and, optionally, stabilizers. In soft capsules, the compound disclosed herein may be dissolved or suspended in suitable liquids, such as fatty oils, liquid paraffin, or liquid polyethylene glycol with or without stabilizers.

Liquid form preparations include solutions, suspensions, and emulsions, for example, water or water/propylene glycol solutions. For parenteral injection, liquid preparations can be formulated in solution in aqueous polyethylene glycol solution.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame, or saccharin. Formulations can be adjusted for osmolarity.

Also included are solid form preparations, which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

Oil suspensions can be formulated by suspending the compound disclosed herein in a vegetable oil, such as arachis oil, olive oil, sesame oil or coconut oil, or in a mineral oil such as liquid paraffin; or a mixture of these. The oil suspensions can contain a thickening agent, such as beeswax, hard paraffin or cetyl alcohol. Sweetening agents can be added to provide a palatable oral preparation, such as glycerol, sorbitol, or sucrose. These formulations can be preserved by the addition of an antioxidant such as ascorbic acid. As an example of an injectable oil vehicle, see Minto, J. Pharmacol. Exp. Ther. 281:93-102, 1997, which is incorporated herein by reference in its entirety for all of its teachings, including without limitation all methods, compounds, compositions, data, and the like, for use with any of the embodiments and disclosure herein. The pharmaceutical formulations disclosed herein can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

The pharmaceutical formulations of the compound disclosed herein can be provided as a salt and can be formed with bases, namely cationic salts such as alkali and alkaline earth metal salts, such as sodium, lithium, potassium, calcium, magnesium, as well as ammonium salts, such as ammonium, trimethyl-ammonium, diethylammonium, and tris-(hydroxymethyl)-methyl-ammonium salts.

The pharmaceutical preparation is preferably in unit dosage form. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted from 0.1 mg to 10000 mg, more typically 1.0 mg to 1000 mg, most typically 10 mg to 500 mg, according to the particular application and the potency of the active component. The composition can, if desired, also contain other compatible therapeutic agents.

The dosage regimen also takes into consideration pharmacokinetics parameters well known in the art, i.e., the rate of absorption, bioavailability, metabolism, clearance, and the like (see, e.g., Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J. Clin. Pharmacol. 24:103-108; the latest Remington's, supra; each of which is incorporated herein by reference in its entirety for all of its teachings, including without limitation all methods, compounds, compositions, data, and the like, for use with any of the embodiments and disclosure herein.). The state of the art allows the clinician to determine the dosage regimen for each individual patient, GR and/or MR modulator and disease or condition treated.

Single or multiple administrations of the compound disclosed herein can be administered depending on the dosage and frequency as required and tolerated by the patient. The formulations should provide a sufficient quantity of active agent to effectively treat the disease state. Thus, in one embodiment, the pharmaceutical formulations for oral administration of the compound disclosed herein is in a daily amount of between about 0.5 to about 30 mg per kilogram of body weight per day. In an alternative embodiment, dosages are from about 1 mg to about 20 mg per kg of body weight per patient per day are used. Lower dosages can be used, particularly when the drug is administered to an anatomically secluded site, such as the cerebral spinal fluid (CSF) space, in contrast to administration orally, into the blood stream, into a body cavity or into a lumen of an organ. Substantially higher dosages can be used in topical administration. Actual methods for preparing formulations including the compound disclosed herein for parenteral administration are known or apparent to those skilled in the art and are described in more detail in such publications as Remington's, supra. See also Nieman, In “Receptor Mediated Antisteroid Action,” Agarwal, et al., eds., De Gruyter, New York (1987), which is incorporated herein by reference in its entirety for all of its teachings, including without limitation all methods, compounds, compositions, data and the like, for use with any of the embodiments and disclosure herein.

In some embodiments, co-administration includes administering one active agent within 0.5, 1, 2, 4, 6, 8, 10, 12, 16, 20, or 24 hours (or any sub-range of time or sub-value of time within a 24 hour period) of a second active agent. Co-administration includes administering two active agents simultaneously, approximately simultaneously (e.g., within about 1, 5, 10, 15, 20, or 30 minutes of each other (or any sub-range of time or sub-value of time from 0-30 minutes for example)), or sequentially in any order. In some embodiments, co-administration can be accomplished by co-formulation, i.e., preparing a single pharmaceutical composition including both active agents. In some embodiments, the active agents can be formulated separately. In some embodiments, the active and/or adjunctive agents may be linked or conjugated to one another. At least one administered dose of drugs can be administered, for example, at the same time. At least one administered dose of the drugs can be administered, for example, within minutes or less than an hour of each other. At least one administered dose of drugs can be administered, for example, at different times, but on the same day, or on different days.

### Kits and Products

Some embodiments relate to kits and products that include the compound of Formula 1 and/or at least one FLT3 inhibitor. For example, the kit or product can include a package or container with a compound disclosed herein. Such kits and products can further include a product insert or label with approved drug administration and indication information, including how to use the compound disclosed herein in combination with a FLT3 inhibitor that is separately provided. The kits can be used in the methods of treating cancer as described herein.

Such kits can include one or more containers or packages, which include one or both combination drugs together in a single container and/or package, or in separate packages/containers. In some instances, the two drugs are separately wrapped, but included in a single package, container, or box. Such kits and products can further include a product insert or label with approved drug administration and indication information, including how to use the compound disclosed herein in combination with a FLT3 inhibitor. The kits can be used in the methods of treating cancer as described herein.

### EXAMPLES

### Example 1—A Phase Lb/2 Trial, Open-Label, Multicenter Study of Compound 1 in Combination with Gilteritinib in Patients with FLT3 Mutant AML

### Study Design

This Phase 1b/2 trial will evaluate compound 1 in combination with gilteritinib in patients with FLT3 mutant AML, which represents 30-40% of all AML, or approximately 61,000 new patients worldwide each year. Only 14% of patients achieve a complete response, nearly all patients experience disease progression, and the median overall survival is less than 10 months. While FLT3 is the most commonly altered gene in AML, alterations along the entire RAS/MAPK pathway are also prevalent, including SHP2, KRAS, NRAS, and BRAF, suggesting dual FLT3 and ERK inhibition or triple FLT3, SHP2, and ERK inhibition may improve the efficacy of gilteritinib monotherapy.

### Part 1 (Dose Escalation)

In Part 1, a weekly recommended dose of compound 1 will be identified in combination with gilteritinib, with a safety assessment as the primary endpoint.

Compound 1 will be evaluated at QW dose levels of 150 mg, 200 mg, and 250 mg, or BID-QW dose levels of 75 mg, 100 mg, and 125 mg in combination with gilteritinib at QD dose level of 120 mg to determine the Recommended Dose (RD) for compound 1.

### Part 2 (Dose Expansion)

In Part 2, the recommended dose regimen of compound 1 in combination with gilteritinib dosed at 120 mg daily will be evaluated in patients with relapsed/refractory AML. The primary endpoint will be an assessment of anti-tumor activity.

### Dosing Schedule

Compound 1 + Gilteritinib in Relapsed/Refractory FLT3m AML

| | |
|---|---|
| Compound 1 QW + Gilteritinib^{a} | Compound 1 BID-QW^{b} + Gilteritinib^{a} |

Dose Escalation

| | |
|---|---|
| Compound 1 150 mg QW + | Compound 1 75 BID-QW + |
| Gilteritinib | Gilteritinib |
| Compound 1 200 mg QW + | Compound 1 100 BID-QW + |
| Gilteritinib | Gilteritinib |
| Compound 1 250 mg QW + | Compound 1 125 BID-QW + |
| Gilteritinib | Gilteritinib |

Dose Expansion

| | |
|---|---|
| Compound 1 QW (RD) + | Compound 1 BID-QW (RD) + |
| Gilteritinib | Gilteritinib |

^{a}Gilteritinib 120 mg PO daily
^{b}BID-QW—Twice a day on a single day each week

### Inclusion Criteria:

Age>18 years.
Willing and able to give written informed consent.
Diagnosis of primary AML or AML secondary to myelodysplastic syndrome (MDS) according to World Health Organization classification.
Relapsed after or refractory to first-line AML therapy.
Positive for FLT3 mutation in bone marrow or whole blood.
Eastern Cooperative Oncology Group performance status ≤2 with no deterioration during screening period.
Adequate hepatic and renal function.
Recovery from non-hematologic AEs associated with prior therapy to baseline CTCAE v5 Grade 0 or 1, except for AEs not considered a safety risk (eg, alopecia or vitiligo).
Able to take oral medication with no medical conditions that prevent swallowing and absorbing oral medications.
Willing to comply with all protocol-required visits, assessments, and procedures.

### Exclusion Criteria

Diagnosis of AML secondary to prior chemotherapy or other neoplasms (except for MDS).
Diagnosis of acute promyelocytic leukemia or BCR-ABL-positive leukemia (chronic myeologenous leukemia in blast crisis).
Clinically active central nervous system leukemia.
Second or later hematologic relapse or prior salvage therapy for refractory disease.
For participants being considered for Compound 1+gilteritinib combination treatment: prior therapy with ERK inhibitor.
Anticancer therapy ≤14 days prior to first dose (except hydroxyurea given for controlling blast count), or ≤5 half-lives prior to first dose, whichever is shorter.
Palliative radiation ≤7 days prior to first dose.
Major surgery within ≤8 days of enrollment.
Contraindication to gilteritinib use as per local label.
Known hypersensitivity to any of the components of Compound 1.
Clinically active infection, requiring systemic therapy
Impaired cardiovascular function or clinically significant cardiovascular disease.
History of thromboembolic or cerebrovascular events ≤6 months prior to first dose.
History of other malignancy ≤3 years prior to first dose.
History of retinal pigment epithelial detachment (RPED), central serous retinopathy, retinal vein occlusion (RVO), or risk factors to RPED or RVO.
History of or clinically active interstitial lung disease (ILD), drug induced ILD, or radiation pneumonitis that required steroid treatment.
Any evidence of severe or uncontrolled systemic disease or evidence of any other significant clinical disorder or laboratory finding that renders the participant inappropriate to participate in the study.
Pregnant or breastfeeding women.

### Example 2

This Example demonstrates the synergistic combination of compound 2 with inhibitors of FLT3 in vitro.

### Methods

Cellular proliferation assay: Cells (2000 cells per well) were plated onto 96-well plates in 100 μl cell culture medium. Cells were treated with compound 2 concentrations varying from 0 to 1 μM and gilteritinib concentrations varying from 0 to 100 nM by using the Tecan D300e Digital Dispenser combination matrix protocol. At day 5, 50 μl of CellTiter-Glo (CTG) reagent (Promega) was added and the plates were incubated for 10 minutes with gentle shaking. After 10 minutes incubation, the luminescent signal was determined according to the provider's instruction (Promega) and combination data was generated by Combenefit software.

Western blotting: Cells were lysed on ice for 10 minutes with Thermo Fisher RIPA lysis buffer with protease and phosphatase inhibitors. The cells were centrifuged at 4° for 10 minutes with a microcentrifuge. The supernatant was transferred to pre-chilled microcentrifuge tube and protein concentration of the lysate was measured using BCA method. Cell lysate supematants of equal-amount of proteins were used for immunoblotting.

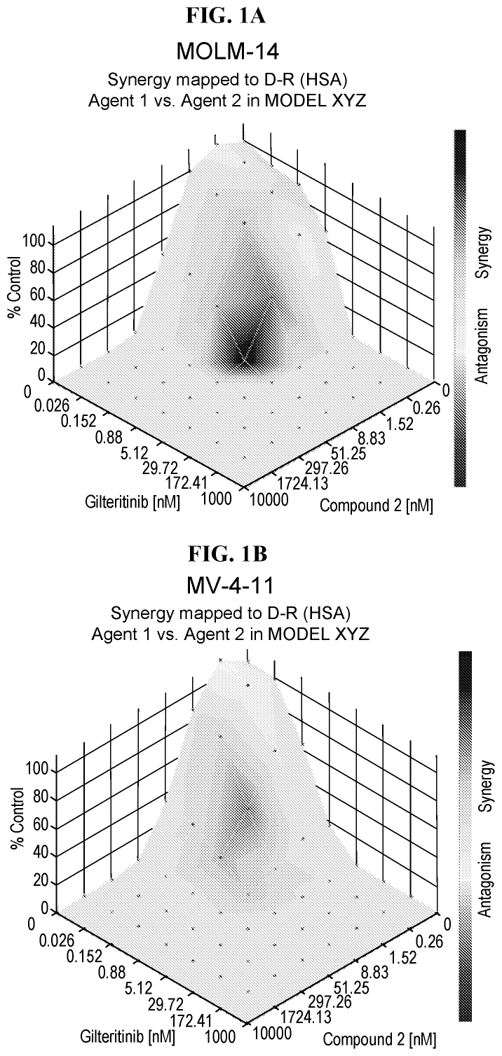

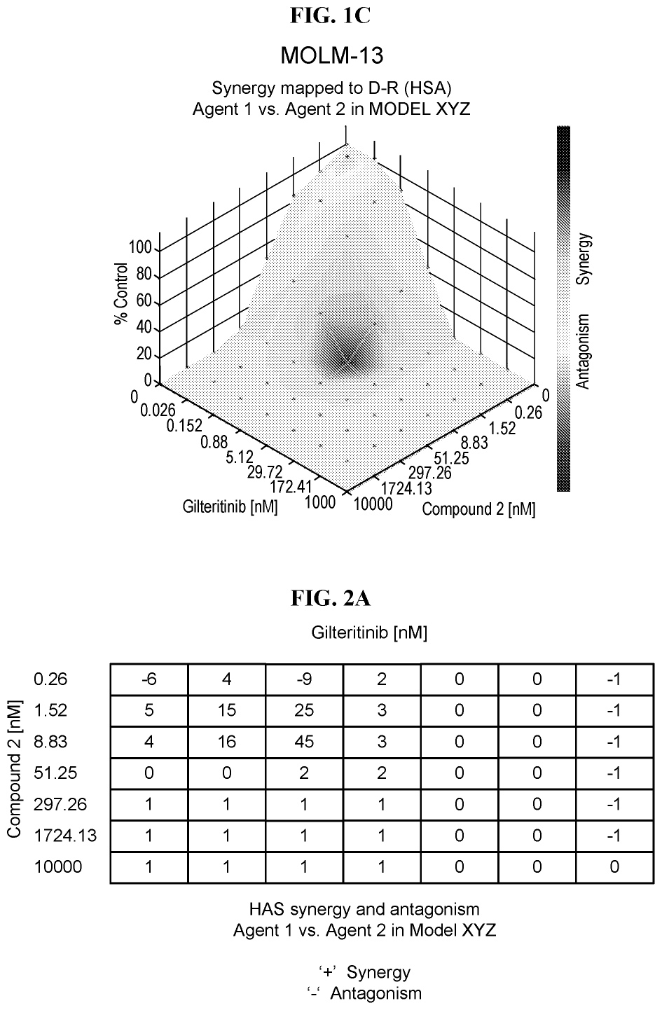

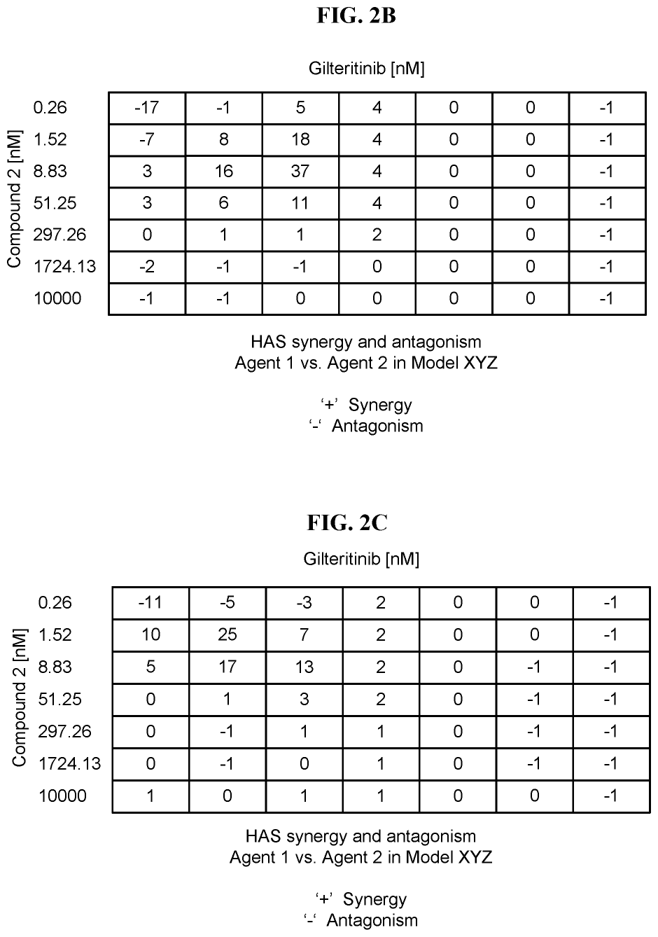

The results of these experiments are indicated in FIGS. 1A-C, 2A/B and 3A/B. Oncogenic signaling via FLT3-ITD mutant proteins was dependent on upstream signaling to drive RAF dimerization and cellular proliferation. SHP2 was an upstream regulator of RAS, transducing upstream signaling via its regulation of the GEF SOS1. SHP2 regulates FLT3-ITD signaling positively in AML cells. Gilteritinib inhibited MAPK signaling in FLT3-ITD mutated tumors, but to maximize therapeutic response and high cure rate, it was to be shown by this Example that the combination of SHP2 inhibition by compound 2 and FLT3-ITD inhibition by gilteritinib would synergistically inhibit cellular proliferation. This was indicated by the results herein in three FLT3-ITD mutated AML cell lines, MOLM-14, MOLM-13, and MV-4-11, were split onto 96 well plates. After overnight incubation, compound 2 and gilteritinib were added to the cells by using the Tecan D300e Digital Dispenser combination matrix protocol. A CellTiter-Glo assay was executed after 5 days of incubation and combination synergy was calculated by the standard HSA model using Combenefit software. The data were represented by calculated synergy results. The higher and positive numbers represent the stronger synergy and negative numbers represent the antagonism. The combination data showed synergistic effects on cellular viability in all three FLT3-ITD mutated cells. Synergy was observed at multiple combination concentrations of compound 2 and gilteritinib. FIG. 1A shows a 3-dimensional colored map indicating that compound 2 and gilteritinib in combination showed synergy in treating FLT3-ITD mutated MOLM-14 AML cells. FIG. 1B is a 3-dimensional colored map indicating that compound 2 and gilteritinib in combination showed synergy in treating FLT3-ITD mutated MV-4-11 AML cells. FIG. 1C is a 3-dimensional colored map indicating that compound 2 and gilteritinib in combination showed synergy in treating FLT3-ITD mutated MOLM-13 AML cells. FIGS. 2a, 2b, 2c show grid plots or numerical data corresponding to FIGS. 1A, 1B, and 1C, respectively. A positive number represents higher synergy and negative numbers represent antagonism in the combination. The numerical data indicate synergy in the combination of compound 2 and gilteritinib in MOLM-14, MV-4-11 and MOLM-13 cells.

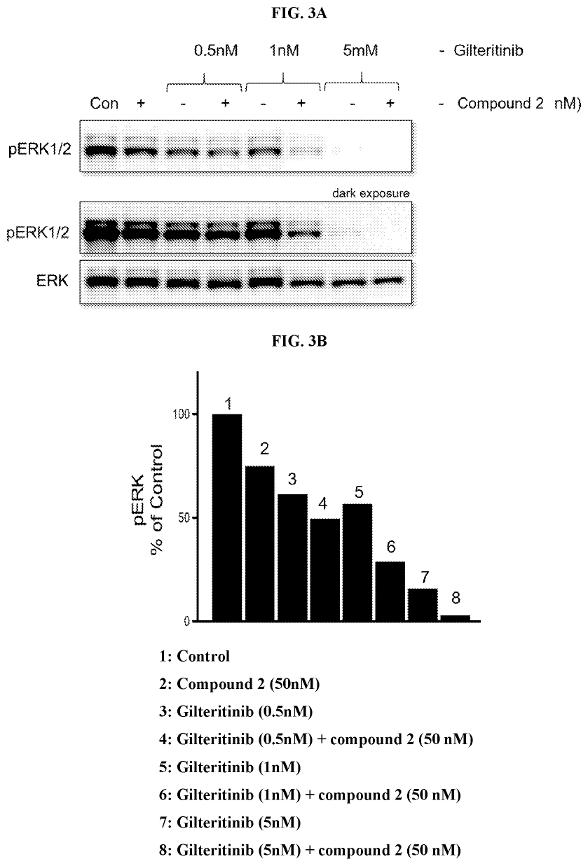

FIG. 3A is a Western blot showing compound 2 and gilteritinib combination induced downregulation of ERK1/2 phosphorylation in MOLM-14 cells. FIG. 3B is a bar plot quantifying the results of FIG. 2A. The cell lines, MOLM14 and MV-4-11 were split onto a 6 well plate and treated with compound 2 and gilteritinib alone, and in combination as indicated (FIGS. 3A/B and 4A/B). After 4 hours treatment, cells were lysed and immunoblotted for phosphorylated ERK1/2. In MOLM-14 cells, the immunoblot results showed that compound 2 inhibited the ERK1/2 phosphorylation about 30% relative to DMSO treated control cells. Gilteritinib (0.5 nM) treatment exhibited a robust inhibition of ERK1/2 phosphorylation and the inhibition was more pronounced when combined with compound 2. An additional pronounced inhibition of pERK1/2 was observed when compound 2 combined with higher concentration of gilteritinib (1 nM) (FIG. 3A). The phosphorylated ERK1/2 bands were quantified by using Bio-Rad Image Lab software and normalized by total ERK. The quantification results showed that compound 2 or gilteritinib treatment decreased the ERK1/2 phosphorylation about 30 to 40% relative to DMSO treated control cells but the combination of compound 2 (50 nM) and gilteritinib (1 nM) showed about 70% inhibition of ERK1/2 phosphorylation in FLT3-ITD mutated MOLM-14 cells (FIG. 3B).

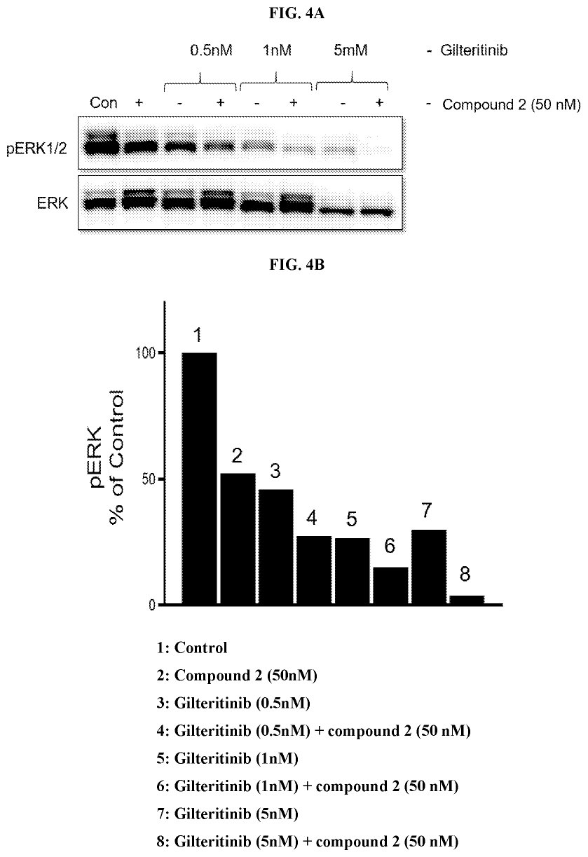

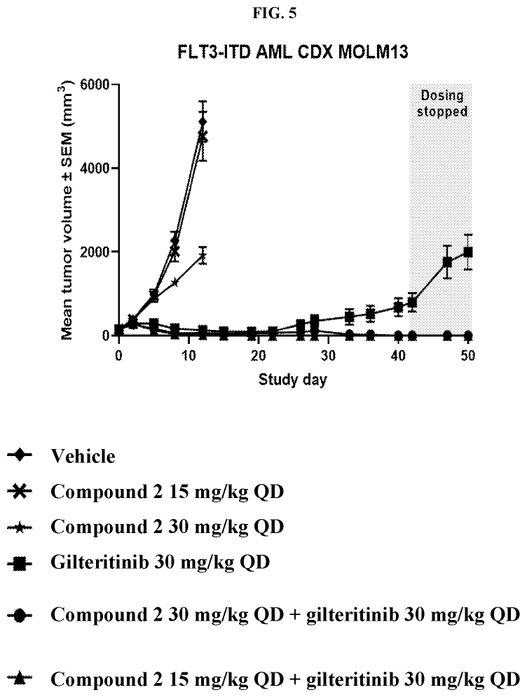

FIG. 4A is a Western blot showing compound 2 and gilteritinib combination induced downregulation of ERK1/2 phosphorylation in MV-4-11 cells. FIG. 4B is a bar plot quantifying the results of FIG. 3A. In MV-4-11 cells, the immunoblot results showed that compound 2 inhibited the ERK1/2 phosphorylation about 50% relative to DMSO treated control cells. Gilteritinib (0.5 nM) treatment exhibited a robust inhibition of ERK1/2 phosphorylation and the inhibition was more pronounced when combined with compound 2. An additional pronounced inhibition of pERK1/2 was observed when compound 2 combined with higher concentration of gilteritinib (1 nM) (FIG. 4A). The phosphorylated ERK1/2 bands were quantified by using Bio-Rad Image Lab software and normalized by total ERK. The quantification results showed that compound 2 or gilteritinib treatment decreased the ERK1/2 phosphorylation about 40 to 50% relative to DMSO treated control cells but the combination of compound 2 (50 nM) and gilteritinib (1 nM) showed about 70% inhibition of ERK1/2 phosphorylation in FLT3-ITD mutated MOLM-14 cells (FIG. 4B).

The above data indicate that the combination of compound 2 and gilteritinib showed synergistic inhibition of ERK1/2 phosphorylation and cellular proliferation across the cell lines screened. The data indicate that compound 2 and gilteritinib can be used to treat tumors harboring oncogenic FLT3-ITD mutation.

Although the foregoing embodiments have been described in some detail by way of illustration and Examples for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

### Example 3—A Phase 1b/2 Trial, Open-Label, Multicenter Study of Compound 2 in Combination with Gilteritinib in Patients with FLT3 Mutant AML

### Study Design

This is a Phase 1b/2, open-label, multicenter master protocol evaluating safety, tolerability, and preliminary efficacy of compound 2 in combination with other cancer therapies in study participants with hematologic malignancies. The study will commence with dose escalation cohorts (compound 2 plus gilteritinib) in study participants with relapsed or refractory (R/R) Feline McDonough sarcoma (FMS)-like tyrosine kinase 3 (FLT3) mutated acute myeloid leukemia (AML). Dose expansion will follow and will evaluate compound 2 drug combinations administered at the RD identified from each respective dose escalation cohort in study participants with R/R FLT-3 mutated AML

### Dose Escalation

Compound 2 will be administered at QD dose levels of 20 mg, 40 mg, 80 mg, and 120 mg, or at BID dose levels of 10 mg, 20 mg, 40 mg, and 60 mg in combination with gilteritinib at QD dose level of 120 mg to study participants with R/R FLT3 mutated AML in sequential ascending doses until unacceptable toxicity, disease progression, or withdrawal of consent.

### Dose Expansion

Compound 2 will be administered at the recommended dose (RD), as determined from the Dose Escalation Arm, in combination with gilteritinib to study participants with R/R FLT3 mutated AML.

### Dosing Schedule

Compound 2 + Gilteritinib in R/R FLT3m AML

| | |
|---|---|
| Compound 2 QD (3/1)^{a} + | Compound 2 BID (3/1)^{b} + |
| Gilteritinib^{c} | Gilteritinib^{c} |

Dose Escalation

| | |
|---|---|
| Compound 2 20 mg QD (3/1) + | Compound 2 10 mg BID (3/1) + |
| Gilteritinib | Gilteritinib |
| Compound 2 40 mg QD (3/1) + | Compound 2 20 mg BID (3/1) + |
| Gilteritinib | Gilteritinib |
| Compound 2 80 mg QD (3/1) + | Compound 2 40 mg BID (3/1) + |
| Gilteritinib | Gilteritinib |
| Compound 2 120 mg QD (3/1) + | Compound 2 60 mg BID (3/1) + |
| Gilteritinib | Gilteritinib |

Dose Expansion

| | |
|---|---|
| Compound 2 QD (3/1) RD + | Compound 2 BID (3/1) RD + |
| Gilteritinib | Gilteritinib |

^{a}QD (3/1)—Daily for 21 days follow by a 7-day break (3 weeks on, 1 week off), on a 28-day cycle
^{b}BID (3/1)—Twice daily for 21 days followed by a 7-day break (3 weeks on, 1 week off) on a 28-day cycle
^{c}Gilteritinib 120 mg PO daily

### Inclusion Criteria:

Age ≥18 years.
Willing and able to give written informed consent.
Diagnosis of primary AML or AML secondary to myelodysplastic syndrome (MDS) according to World Health Organization classification.
Relapsed after or refractory to first-line AML therapy.
Positive for FLT3 mutation in bone marrow or whole blood.
Eastern Cooperative Oncology Group performance status ≤2 with no deterioration during screening period.
Adequate hepatic and renal function.
Recovery from non-hematologic AEs associated with prior therapy to baseline CTCAE v5 Grade 0 or 1, except for AEs not considered a safety risk (e.g., alopecia or vitiligo).
Able to take oral medication with no medical conditions that prevent swallowing and absorbing oral medications.
Willing to comply with all protocol-required visits, assessments, and procedures.

### Exclusion Criteria:

Diagnosis of AML secondary to prior chemotherapy or other neoplasms (except for MDS).
Diagnosis of acute promyelocytic leukemia or BCR-ABL-positive leukemia (chronic myelogenous leukemia in blast crisis).
Clinically active central nervous system leukemia.
Second or later hematologic relapse or prior salvage therapy for refractory disease.
For participants being considered for compound 2+gilteritinib combination treatment: prior therapy with ERK inhibitor.
Anticancer therapy ≤14 days prior to first dose (except hydroxyurea given for controlling blast count), or ≤5 half-lives prior to first dose, whichever is shorter.
Palliative radiation ≤7 days prior to first dose.
Major surgery within 28 days of enrollment.
Contraindication to gilteritinib use as per local label.
Known hypersensitivity to any of the components of compound 2.
Clinically active infection, requiring systemic therapy
Impaired cardiovascular function or clinically significant cardiovascular disease.
History of thromboembolic or cerebrovascular events ≤6 months prior to first dose.
History of other malignancy ≤3 years prior to first dose.
History of retinal pigment epithelial detachment (RPED), central serous retinopathy, retinal vein occlusion (RVO), or risk factors to RPED or RVO.
History of or clinically active interstitial lung disease (ILD), drug induced ILD, or radiation pneumonitis that required steroid treatment.
Any evidence of severe or uncontrolled systemic disease or evidence of any other significant clinical disorder or laboratory finding that renders the participant inappropriate to participate in the study.
Pregnant or breastfeeding women.

### Example 4—In Vivo Study of Compound 2 Alone, Gilteritinib Alone, and Compound 2+Gilteritinib Combination in a FLT3-ITD Mutated AML Subcutaneous CDX Model MOLM-13

### Vehicle/Control Article

The vehicle/control article, 100 mM acetic acid in deionized water, with pH adjustment to 4.8-5.0, was prepared and stored under ambient conditions throughout the 42-day administration in mice.

### Formulation of Test Article

The test article, compound 2, was freshly prepared in vehicle of 100 mM acetic buffer weekly and stored under ambient conditions. The combination agent gilteritinib was prepared weekly in vehicle of 0.5% Methyl Cellulose & 0.1% Tween 80 Solution weekly and stored under ambient conditions.

### Animals

Female Balb/c nude mice were between 6-8 weeks of age at the time of implantation. Mice were hosted in a special pathogen-free (SPF) environment of the vivarium facility and acclimated to their new environment for at least 3 days prior to initiation of any experiments according to IACUC protocol.

### Preparation of Xenograft Model

MOLM-13 was AML cell line that harbored a FLT3-ITD mutation. MOLM-13 cells were cultured in medium containing RPMI1640 plus 10% Fetal Bovine Serum (FBS) and 1% Antibiotic-Antimycotic (AA), at 37° C. in an atmosphere of 5% CO₂ in air. The medium was renewed every 2 to 3 days and tumor cells were routinely sub-cultured at a confluence of 80-90% by trypsin-EDTA. Cells growing in an exponential growth phase were harvested and counted for inoculation. MOLM-13 tumor cells (passage 6) were implanted into mice subcutaneously. 200 μL cell suspensions containing 5×10⁶ tumor cells mixed with 50% Matrigel were subcutaneously implanted into the right flank of mouse using a syringe. Totally 180 mice were implanted in this study. Animal health and tumor growth were monitored daily. Tumor volume was measured twice a week by caliper when tumors were palpable and measurable. When tumor volumes reached a mean of 151 mm³ (range of 80-276 mm³) at day 8 post subcutaneous implantation, tumor-bearing mice were randomized into different groups with 8 mice in each group. The randomization date was denoted as treatment day 0.

### Treatment

Treatment started on the day after randomization. The treatment start day was denoted as treatment day 1. Mice were treated as shown below in Table 1. The vehicle control group, compound 2 monotherapy groups were terminated on treatment day 12 due to rapid tumor growth. The treatment of gilteritinib monotherapy group and all combination treatment groups were extended to day 42 after randomization, and animal body weight and tumor measurement of gilteritinib monotherapy group and all combination treatment groups were extended to day 50 after randomization.

TABLE 1
Treatment Schedule of Compounds
and Combinations in MOLM-13

| | | Dose | | |
|---|---|---|---|---|
| | Dose | Volume¹ | Dose | |
| Treatment | (mg/kg) | (mL/kg) | Route | Schedule² |
| Vehicle | — | 5 | PO | BID × 12 |
| Gilteritinib | 30 | 5 | PO | QD × 42 |
| Compound 2 | 15 | 5 | PO | QD × 12 |
| Compound 2 | 30 | 5 | PO | QD × 12 |
| Compound 2 + | 15 + 30 | 5 + 5 | PO + PO | QD + QD × 42 |
| Gilteritinib | | | | |
| Compound 2 + | 30 + 30 | 5 + 5 | PO + PO | QD + QD × 42 |
| Gilteritinib | | | | |

### Results and Conclusion

As shown in FIG. 5, treatment with the combination of compound 2+gilteritinib showed durable tumor growth inhibition in vivo in FLT3-ITD mutated AML subcutaneous CDX model MOLM-13 as compared to their respective monotherapies.

### Example 5—In Vivo Study of Compound 2 Alone, Gilteritinib Alone, and Compound 2+Gilteritinib Combination in a FLT3-ITD Mutated AML Subcutaneous CDX Model MOLM-13

### Vehicle/Control Article

The vehicle/control article, 100 mM acetic acid in deionized water, with pH adjustment to 4.8-5.0, was prepared and stored under ambient conditions throughout the 28-day administration in mice.

### Formulation of Test Article

The test article, compound 2 was freshly prepared in vehicle of 100 mM acetic buffer weekly and stored under ambient conditions. The combination agent gilteritinib was prepared weekly in vehicle of 0.5% Methyl Cellulose & 0.1% Tween 80 Solution weekly and stored under ambient conditions.

### Animals

Female Balb/c nude mice were between 6-8 weeks of age at the time of implantation. Mice were hosted in a special pathogen-free (SPF) environment of the vivarium facility and acclimated to their new environment for at least 3 days prior to initiation of any experiments according to IACUC protocol.

### Preparation of Orthotopic Model

MOLM-13 was AML cell line that harbored a FLT3-ITD mutation. For generating MOLM-13-luc cell line, MOLM-13 cells were transfected with GFP-LUC-puro Lentivirus (HBLV-1012, HANBIO) and then selected with 1 μg/mL puromycin. MOLM-13-luc cells were cultured in medium containing RPMI1640 plus 10% Fetal Bovine Serum (FBS) and 1% Antibiotic-Antimycotic (AA), at 37° C. in an atmosphere of 5% CO₂ in air. The medium was renewed every 2 to 3 days and tumor cells were routinely sub-cultured at a confluence of 80-90%. Cells growing in an exponential growth phase were harvested and counted for inoculation. MOLM-13-luc tumor cells were implanted via tail vein. 200 μL cell suspensions containing 2×10⁶ tumor cells were implanted into the mouse via tail vein using a syringe. Animal health were monitored daily. Imaging of Bioluminescence from tumor cells in whole mouse body was measured twice a week by Xenogen machine (PerkinElmer IVIS Lumina III). When bioluminescence reached a mean of 2.10E+06 photons/second (range of 9.86E+05-3.84E+06 photons/second) at day 5 post tail vein implantation, AML orthotopic tumor-bearing mice were randomized into study groups with 8 mice in each group. The randomization date was denoted as treatment day 0.

### Treatment

Treatment started on the day after randomization. The treatment start day was denoted as treatment day 1. Mice were treated as shown in Table 2 below.

TABLE 2
Treatment Schedule of Compounds
and Combinations in MOLM-13-Luc

| | | Dose | | |
|---|---|---|---|---|
| | Dose | Volume | Dose | |
| Treatment | (mg/kg) | (mL/kg) | Route | Schedule |
| Vehicle | — | 5 | PO | BID × 10 |
| Compound 2 | 15 | 5 | PO | QD × 10 |
| Compound 2 | 30 | 5 | PO | QD × 17 |
| Gilteritinib | 30 | 5 | PO | QD × 28 |
| Compound 2 + | 15 + 30 | 5 + 5 | PO + PO | QD + QD × 28 |
| Gilteritinib | | | | |

### Results and Conclusion

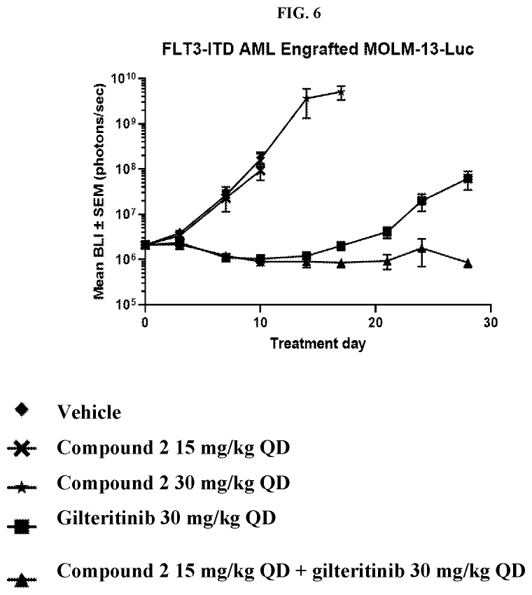

As shown in FIG. 6, treatment with the combination of compound 2+gilteritinib achieved superior tumor growth inhibition in vivo in FLT3-ITD mutated AML subcutaneous CDX model MOLM-13-Luc as compared to their respective monotherapies.

1. A method of treating cancer in a subject in need thereof, the method comprising: administering to the subject in need thereof a therapeutically effective amount of
(i) compound 1: 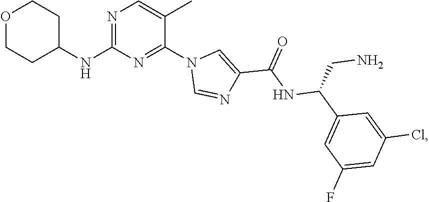 [SMILES: Cc1cnc(NC2CCOCC2)nc1-n1cnc(C(=O)N[C@H](CN)c2cc(F)cc(Cl)c2)c1]
or a pharmaceutically acceptable salt thereof; and
(ii) a FLT3 inhibitor.

2. A method of treating cancer in a subject in need thereof, the method comprising: administering to the subject in need thereof a therapeutically effective amount of
(ii) compound 2 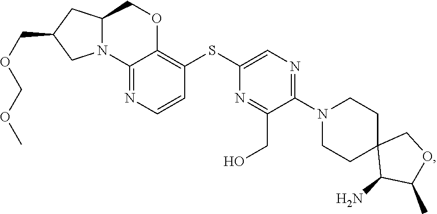 [SMILES: COCOC[C@H]1C[C@H]2COc3c(Sc4cnc(N5CCC6(CC5)CO[C@@H](C)[C@H]6N)c(CO)n4)ccnc3N2C1]
or a pharmaceutically acceptable salt thereof; and
(ii) a FLT3 inhibitor.

3. The method of claim 1 or 2, wherein the dosing of the FLT3 inhibitor is in a range from about 1 mg/day to about 500 mg/day.

4. The method of any one of claims 1-3, wherein the FLT3 inhibitor is crenolanib, gilteritinib, ibrutinib, lestaurtinib, midostaurin, ponatinib, quizartinib, sorafenib, sunitinib, or tandutinib.

5. The method of any one of claims 1-4, wherein the FLT3 inhibitor is gilteritinib.

6. The method of claim 5, wherein gilteritinib is administered in an amount that is about 120 mg/day.

7. The method of claim 5 or 6, wherein gilteritinib is administered once or twice daily.

8. A method of treating cancer in a subject in need thereof, the method comprising: administering to the subject in need thereof a therapeutically effective amount of
(i) compound 1: 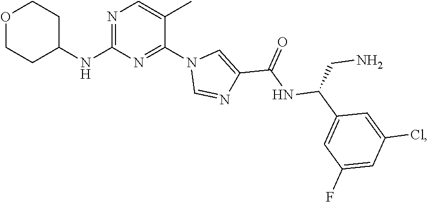 [SMILES: Cc1cnc(NC2CCOCC2)nc1-n1cnc(C(=O)N[C@H](CN)c2cc(F)cc(Cl)c2)c1]
or a pharmaceutically acceptable salt thereof; and
(ii) gilteritinib.

9. The method of any one of claims 1 or 3-8, wherein the pharmaceutically acceptable salt of compound 1 is the mandelic acid salt.

10. A method of treating cancer in a subject in need thereof, the method comprising: administering to the subject in need thereof a therapeutically effective amount of
(i) compound 2: 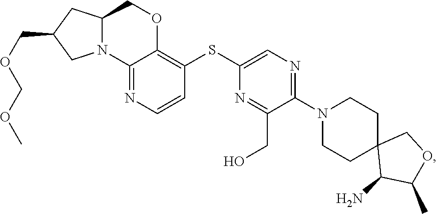 [SMILES: COCOC[C@H]1C[C@H]2COc3c(Sc4cnc(N5CCC6(CC5)CO[C@@H](C)[C@H]6N)c(CO)n4)ccnc3N2C1]
or a pharmaceutically acceptable salt thereof; and
(ii) gilteritinib.

11. The method of any one of claims 1-10, wherein the cancer is a mitogen-activated protein kinase (MAPK) pathway driven cancer.

12. The method of any one of claims 1-10, wherein the cancer is a BRAF-driven cancer, HRAS-driven cancer, or a NRAS-driven cancer.

13. The method of any one of claims 1-10, wherein the cancer comprises at least one cancer cell driven by deregulated ERK.

14. The method of any one of claims 1-10, wherein the cancer has at least one mutation in RAS.

15. The method of any one of claims 1-10, wherein the cancer has at least one mutation in RAF.

16. The method of any one of claims 1-10, wherein the cancer has at least one mutation in MEK.

17. The method of any one of claims 1-10, wherein the cancer has a G12C KRAS mutation.

18. The method of any one of claims 1-10, wherein the cancer has a G12D KRAS mutation.

19. The method of any one of claims 1-10, wherein the cancer has a G12S KRAS mutation.

20. The method of any one of claims 1-10, wherein the cancer has a G12V KRAS mutation.

21. The method of any one of claims 1-10, wherein the cancer has a G13D KRAS mutation.

22. The method of any one of claims 1-10, wherein the cancer has a Q16H KRAS mutation.

23. The method of any one of claims 1-10, wherein the cancer has a Q16K KRAS mutation.

24. The method of any one of claims 1-10, wherein the cancer has a Q61R NRAS mutation.

25. The method of any one of claims 1-10, wherein the cancer is a BRAF V600E or V600K mutant tumor.

26. The method of any one of claims 1-10, wherein the cancer is a MAPKm/MAPKi-naïve pancreatic cancer.

27. The method of any one of claims 1-10, wherein the cancer comprises one or more EGFR mutation selected from the group consisting of EGFR gene copy gain, EGFR gene amplification, chromosome 7 polysomy, L858R, exon 19 deletions/insertions, L861Q, G719C, G719S, G719A, V765A, T783A, exon 20 insertions, EGFR splice variants (Viii, Vvi, and Vii), A289D, A289T, A289V, G598A, G598V, T790M, and C797S.

28. The method of any one of claims 1-10, wherein the cancer comprises one or more EGFR mutation selected from the group consisting of L858R, exon 19 deletion, and T790M.

29. The method of any one of claims 1-28, wherein the cancer is a liquid tumor.

30. The method of claim 29, wherein the liquid tumor is leukemia.

31. The method of claim 30, wherein the leukemia is acute myeloid leukemia (AML).

32. The method of claim 31, wherein the AML is relapsed and/or refractory AML.

33. The method of claim 31, wherein the AML is a FLT3 mutant AML.

34. The method of any one of claims 1-28, wherein the cancer is a hematologic cancer.

35. The method of any one of claims 1-28, wherein the cancer acute myeloid leukemia.

36. The method of any one of claims 1-28, wherein the cancer is colorectal cancer.

37. The method of any one of claims 1-28, wherein the cancer is an adenocarcinoma of the lung or colon.

38. The method of any one of claims 1-28, wherein the cancer is cutaneous melanoma.

39. The method of any one of claims 1-28, wherein the cancer is breast invasive ductal carcinoma.

40. The method of any one of claims 1-39, wherein compound 1, or a pharmaceutically acceptable salt thereof, is administered in an amount that is between about 25 mg/day and about 300 mg/day.

41. The method of any one of claims 1-40, wherein compound 1, or a pharmaceutically acceptable salt thereof, is administered in an amount that is between 25 mg/day and 150 mg/day.

42. The method of any one of claims 1-41, wherein compound 1, or a pharmaceutically acceptable salt thereof, is administered in an amount that is about 25 mg/day, about 50 mg/day, about 75 mg/day, about 100 mg/day, about 125 mg/day, about 150 mg/day, about 175 mg/day, about 200 mg/day, about 225 mg/day, or about 250 mg/day.

43. The method of any one of claims 1-42, wherein compound 1, or a pharmaceutically acceptable salt thereof, is administered in an amount that is about 25 mg/day, about 50 mg/day, about 100 mg/day, or about 150 mg/day.

44. The method of any one of claims 2-39, wherein compound 2, or a pharmaceutically acceptable salt thereof, is administered in an amount that is between 20 mg/day and 400 mg/day.

45. The method of any one of claims 2-39, wherein compound 2, or a pharmaceutically acceptable salt thereof, is administered in an amount that is between 10 mg/day and 100 mg/day.

46. The method of any one of claims 2-39, wherein compound 2, or a pharmaceutically acceptable salt thereof, is administered in an amount that is between 20 mg/day and 80 mg/day.

47. The method of any one of claims 1-46, wherein compound 1 or compound 2, or a pharmaceutically acceptable salt thereof, is administered once a day (QD).

48. The method of any one of claims 1-46, wherein compound 1 or compound 2, or a pharmaceutically acceptable salt thereof, is administered twice a day (BID).

49. The method of any one of claims 1-46, wherein compound 1 or compound 2, or a pharmaceutically acceptable salt thereof, is administered three times a day (TID).

50. The method of any one of claims 1-49, wherein compound 1 or compound 2, or a pharmaceutically acceptable salt thereof, is administered once a week.

51. The method of any one of claims 1-49, wherein compound 1 or compound 2, or a pharmaceutically acceptable salt thereof, is administered twice a week.

52. The method of any one of claims 1-39, wherein compound 1, or a pharmaceutically acceptable salt thereof, is administered in an amount that is between about 25 mg and about 300 mg twice a day, once a week (BID-QW).

53. The method of any one of claims 1-39, wherein compound 1, or a pharmaceutically acceptable salt thereof, is administered in an amount that is between about 25 mg and about 250 mg twice a day, once a week (BID-QW).

54. The method of any one of claims 1-39, wherein compound 1, or a pharmaceutically acceptable salt thereof, is administered in an amount that is between about 25 mg and about 150 mg twice a day, once a week (BID-QW).

55. The method of any one of claims 1-39, wherein compound 1, or a pharmaceutically acceptable salt thereof, is administered in an amount that is about 25 mg, 50 mg, about 75 mg, about 100 mg, about 125 mg, about 150 mg, about 175 mg, about 200 mg, about 225 mg, or about 250 mg twice a day, once a week (BID-QW).

56. The method of any one of claims 1-39, wherein compound 1, or a pharmaceutically acceptable salt thereof, is administered in an amount that is about 25 mg, 50 mg, about 100 mg, about 125 mg, or about 150 mg twice a day, once a week (BID-QW).

57. The method of any one of claims 1-39, wherein compound 1, or a pharmaceutically acceptable salt thereof, is administered in an amount that is about 125 mg twice a day, once a week (BID-QW).

58. The method of any one of claims 1-57, wherein compound 1 or compound 2, or a pharmaceutically acceptable salt thereof, is administered for at least one 28-day cycle.

59. The method of any one of claims 1-57, wherein compound 1 or compound 2, or a pharmaceutically acceptable salt thereof, is administered on day 1, day 8, day 15, and day 22 of a 28-day cycle.

60. The method of any one of claims 1-57, wherein compound 1 or compound 2, or a pharmaceutically acceptable salt thereof, is administered on day 1, day 8, day 15 of a 28-day cycle.

61. The method of any one of claims 1-57, wherein compound 1 or compound 2, or a pharmaceutically acceptable salt thereof, is administered for 2 weeks on and 1 week off (21 day schedule).

62. The method of any one of claims 1-57, wherein compound 1 or compound 2, or a pharmaceutically acceptable salt thereof, is administered for 3 weeks on and 1 week off (28 day schedule).

63. The method of any one of claims 1-57, wherein compound 1 or compound 2, or a pharmaceutically acceptable salt thereof, is administered three times a week (D1D3D5 TIW).

64. The method of any one of claims 1-57, wherein compound 1 or compound 2, or a pharmaceutically acceptable salt thereof, is administered twice a day/twice a week (BID-D1D2-BIW).

65. The method of any one of claims 1-64, wherein compound 1 or compound 2, or a pharmaceutically acceptable salt thereof, is administered orally.

66. The method of any one of claims 1-65, wherein the dose of the FLT3 inhibitor is less than the dose required for a monotherapy with the FLT3 inhibitor.

67. The method of any one of claims 1-65, wherein the dose of compound 1 or compound 2 is less than the dose required for a monotherapy with compound 1 or compound 2.

68. The method of any one of claims 1-67, wherein the method further comprises administering an additional MAPK pathway inhibitor.

69. The method of claim 68, wherein the additional MAPK pathway inhibitor is a KRAS inhibitor, NRAS inhibitor, HRAS inhibitor, PDGFRA inhibitor, PDGFRB inhibitor, MET inhibitor, FGFR inhibitor, ALK inhibitor, ROS1 inhibitor, TRKA inhibitor, TRKB inhibitor, TRKC inhibitor, EGFR inhibitor, IGFR1R inhibitor, GRB2 inhibitor, SOS inhibitor, ARAF inhibitor, BRAF inhibitor, RAF1 inhibitor, MEK1 inhibitor, MEK2 inhibitor, c-Mycv, CDK4/6, inhibitor CDK2 inhibitor, FLT3 inhibitor, or ERK1/2 inhibitor.

70. A kit comprising compound 1, or a pharmaceutically acceptable salt thereof, and a FLT3 inhibitor.

71. The kit of claim 70, wherein compound 1, or a pharmaceutically acceptable salt thereof, and the FLT3 inhibitor are in separate packages.

72. A kit comprising compound 2, or a pharmaceutically acceptable salt thereof, and a FLT3 inhibitor.

73. The kit of claim 72, wherein compound 2, or a pharmaceutically acceptable salt thereof, and the FLT3 inhibitor are in separate packages.

74. The kit of any one of claims 70-73, wherein the kit further comprises instructions to administer the contents of the kit to a subject for the treatment of cancer.

75. The kit of any one of claims 70-74, wherein the FLT inhibitor is tandutinib, ibrutinib, sorafenib, quizartinib, ponatinib, sunitinib, lestaurtinib, midostaurin, crenolanib, or gilteritinib.

76. The kit of any one of claims 70-75, wherein the FLT inhibitor is gilteritinib.
